(19) United States
(12) Patent Application Publication
(10) Pub. No.: US 2002/0039746 A1
(43) Pub. Date: Apr. 4, 2002

(54) Methods for identifying peripheral benzodiazepine receptor binding agents

(76) Inventors: Eric Richard Sjoberg, San Diego, CA (US); Gonul Velicelebi, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/765,103

(22) Filed: Jan. 16, 2001

Related U.S. Application Data
(60) Provisional application No. 60/176,180, filed on Jan. 14, 2000.

(51) Int. Cl. G01N 33/53; C12N 5/08; A61K 31/5513

(52) U.S. Cl. 435/7.1; 435/368; 514/221

(57) ABSTRACT

The invention provides methods for screening for agents that modulate mitochondrial membrane potential. Such agents generally bind to a peripheral benzodiazepine receptor and may be detected by direct binding assays or by indirect or functional assays. Agents identified using the screens provided herein have application in the prevention and treatment of a variety of diseases associated with abnormal mitochondrial function.

### CROSS-REFERENCES TO RELATED APPLICATIONS

This application claims the benefit of U.S. Provisional Application No. 60/176,180 filed Jan. 14, 2000, which is incorporated herein by reference in its entirety.

### TECHNICAL FIELD

The invention relates generally to assays for screening for agents that alter mitochondrial function. More specifically, the invention relates to compositions and screening methods for use in identifying agents that bind a peripheral benzodiazepine receptor, including cell lines that constitutively or inducibly overexpress a peripheral benzodiazepine receptor.

### BACKGROUND OF THE INVENTION

Mitochondria are organelles that are the main energy source in cells of higher organisms. These organelles provide direct and indirect biochemical regulation of a wide array of cellular respiratory, oxidative and metabolic processes, including metabolic energy production, aerobic respiration and intracellular calcium regulation. For example, mitochondria are the site of electron transport chain (ETC) activity, which drives oxidative phosphorylation to produce metabolic energy in the form of adenosine triphosphate (ATP), and which also underlies a central mitochondrial role in intracellular calcium homeostasis. These processes require the maintenance of a mitochondrial membrane electrochemical potential, and defects in such membrane potential can result in a variety of disorders.

In addition to their role in energy production in growing cells, mitochondria (or at least mitochondrial components) participate in programmed cell death (PCD), also known as apoptosis (see Newmeyer et al., Cell 79:353-364, 1994; Liu et al., Cell 86:147-157, 1996). Apoptosis is apparently required for normal development of the nervous system and functioning of the immune system. Some disease states are associated with insufficient apoptosis (e.g., cancer and autoimmune diseases) or excessive levels of apoptosis (e.g., stroke and neurodegeneration). For general review of apoptosis, and the role of mitochondria therein, see Green and Reed, Science 281:1309-1312, 1998; Green, Cell 94:695-698, 1998 and Kromer, Nature Medicine 3:614-620, 1997.

Mitochondria contain an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix (for review, see, e.g., Ernster et al., 1981 J. Cell Biol. 91:227s.) While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (greater than about 10 kD) molecules.

Four of the five multisubunit protein complexes (Complexes I, III, IV and V) that mediate ETC activity are localized to the inner mitochondrial membrane. The remaining ETC complex (Complex II) is situated in the matrix. In at least three distinct chemical reactions known to take place within the ETC, protons are moved from the mitochondrial matrix, across the inner membrane, to the intermembrane space. This disequilibrium of charged species creates an electrochemical membrane potential of approximately 220 mV referred to as the “protonmotive force” (PMF). The PMF, which is often represented by the notation Δp, corresponds to the sum of the electric potential (ΔΨm) and the pH differential (ΔpH) across the inner membrane according to the equation

Δp=ΔΨm−ZΔpH

wherein Z stands for −2.303 RT/F. The value of Z is −59 at 25° C. when Δp and ΔΨm are expressed in mV and ΔpH is expressed in pH units (see, e.g., Ernster et al., J. Cell Biol. 91:227s, 1981 and references cited therein).

ΔΨm provides the energy for phosphorylation of adenosine diphosphate (ADP) to yield ATP by ETC Complex V, a process that is coupled stoichiometrically with transport of a proton into the matrix. ΔΨm is also the driving force for the influx of cytosolic Ca²⁺ into the mitochondrion. Under normal metabolic conditions, the inner membrane is impermeable to proton movement from the intermembrane space into the matrix, leaving ETC Complex V as the sole means whereby protons can return to the matrix. When, however, the integrity of the inner mitochondrial membrane is compromised, as occurs during mitochondrial permeability transition (MPT) that accompanies certain diseases associated with altered mitochondrial function, protons are able to bypass the conduit of Complex V without generating ATP, thereby uncoupling respiration. During MPT, ΔΨm collapses and mitochondrial membranes lose the ability to selectively regulate permeability to solutes both small (e.g., ionic Ca²⁺, Na⁺, K⁺ and H⁺) and large (e.g., proteins). “Altered mitochondrial function” may refer to any condition or state, including those that accompany a disease associated with altered mitochondrial function, where any structure or activity that is directly or indirectly related to a mitochondrial function has been changed in a statistically significant manner relative to a control or standard. Altered mitochondrial function may have its origin in extramitochondrial structures or events as well as in mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

Additionally, altered mitochondrial function may include altered respiratory, metabolic or other biochemical or biophysical activity in one or more cells of a biological sample or a biological source. As non-limiting examples, markedly impaired ETC activity may be related to altered mitochondrial function, as may be generation of increased reactive oxygen species (ROS) or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of altered mitochondrial function. These and other non-limiting examples of altered mitochondrial function are contemplated by the present invention.

Without wishing to be bound by theory, altered mitochondrial function may be related, inter alia, to altered intracellular calcium regulation that may, for example, accompany loss of mitochondrial membrane electrochemical potential by intracellular calcium flux, by mechanisms that include free radical oxidation, defects in transmitochondrial membrane shuttles and transporters such as the adenine nucleotide transporter or the malate-aspartate shuttle, by defects in ATP biosynthesis, by impaired association with porin of hexokinases and/or other enzymes or by other events. Altered intracellular calcium regulation and/or collapse of mitochondrial inner membrane potential may result from direct or indirect effects of mitochondrial genes, gene products or related downstream mediator molecules and/or extramitochondrial genes, gene products or related downstream mediators, or from other known or unknown causes. Thus, an “indicator of altered mitochondrial function” may be any detectable parameter that directly relates to a condition, process, pathway, dynamic structure, state or other activity involving mitochondria and that permits detection of altered mitochondrial function in a biological sample from a subject or biological source. According to non-limiting theory, altered mitochondrial function therefore may also include altered mitochondrial permeability to calcium or to mitochondrial molecular components involved in apoptosis (e.g., cytochrome c), or other alterations in mitochondrial respiration.

Loss of mitochondrial membrane electrochemical potential may therefore be the result of mechanisms such as free radical oxidation, or may be due to direct or indirect effects of mitochondrial and/or extramitochondrial gene products. Loss of mitochondrial potential appears to be a critical event in the progression of diseases associated with altered mitochondrial function, including degenerative diseases such as Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome. Diseases associated with altered mitochondrial function thus include these and other diseases in which one or more levels of an indicator of altered mitochondrial function differ in a statistically significant manner from the corresponding indicator levels found in clinically normal subjects known to be free of a presence or risk or such disease.

Defective mitochondrial activity may alternatively or additionally result in the generation of highly reactive free radicals that have the potential of damaging cells and tissues. These free radicals may include reactive oxygen species (ROS) such as superoxide, peroxynitrite and hydroxyl radicals, and potentially other reactive species that may be toxic to cells. For example, oxygen free radical induced lipid peroxidation is a well established pathogenic mechanism in central nervous system (CNS) injury such as that found in a number of degenerative diseases, and in ischemia (i.e., stroke). Mitochondrial involvement in the apoptotic cascade has been identified, for example mitochondrial release of cytochrome c, and may therefore be a factor in neuronal death that contributes to the pathogenesis of certain neurodegenerative (i e., CNS) diseases.

The peripheral benzodiazepine receptor (PBzR or PBR) is an 18 kDa protein that has been detected on the outer mitochondrial membrane of many cell types. Based on localization of PBzR to sites of contact between the inner and outer mitochondrial membrane, and its apparent association with certain mitochondrial membrane proteins such as the voltage dependent anion channel (VDAC, also known as porin) and the adenine nucleotide translocator (ANT), PBzR has been implicated in various mitochondrial processes, including cholesterol translocation across membranes, protection against ROS damage and regulation of ion channels (Carayon et al., 1996 Blood 87:3170; Papadopoulos et al., 1997 J. Biol. Chem. 51:32129; Tsankova et al., 1995 Eur. J Pharmacol. 294:601).

PK11195, an isoquinolone compound that is a ligand of PBR, enhances the apoptogenic effects of several known apoptogenic compounds in several cell types, but does not by itself induce apoptosis (Hirsch et al., 1998 Exp. Cell Res. 241:426; Ravagnan et al., 1999 Oncogene 18:2537). PK11195 also appears to counteract anti-apoptotic, cytoprotective effects of the Bcl-2 proto-oncogene product, suggesting a PBzR role in regulating apoptosis, which is known to be under the control of significant mitochondrial regulation (see, e.g., Green et al., 1998 Science 281:1309 and references cited therein). However, contributions of PBR to mitochondrial regulation of biological processes have been difficult to discern, in part because current evaluation of natural or recombinantly induced PBR expression suggests that PBR is not abundantly expressed.

Thus, while numerous mitochondrial functions are altered in various disease states, there remains a clear need for improved understanding of specific mitochondrial molecular mechanisms that underlie disease processes, including those that involve PBR. To provide improved therapies for such diseases, agents that alter mitochondrial function may be beneficial, and assays to specifically detect such agents are needed. The present invention fulfills these needs and further provides other related advantages.

### SUMMARY OF THE INVENTION

The present invention is directed in part to methods for identifying agents that alter mitochondrial function. Compositions and methods are provided for screening assays, including high throughput screens, which employ cells that overexpress a PBR that in certain embodiments are neuronal cells and in certain other embodiments are hematopoietic cells, including permeabilized cells that overexpress a PBR or mitochondria derived therefrom. In certain embodiments the invention relates to a method that comprises screening for an agent that alters (e.g., increases or decreases in a statistically significant manner) the binding interaction between a PBR and a PBR ligand. In certain other embodiments the invention relates to a method that comprises screening for an agent that alters mitochondrial function by comparing, in the absence and presence of a candidate agent, mitochondrial membrane potential, apoptosis, Bcl-2 binding to a Bcl-2 ligand or binding of a PBR ligand to a PBR.

Accordingly, it is an aspect of the invention to provide a method of screening for an agent that binds a peripheral benzodiazepine receptor, comprising the steps of (a) contacting a sample comprising a mitochondrion from a cell that overexpresses a peripheral benzodiazepine receptor with a peripheral benzodiazepine receptor ligand and a candidate agent; and (b) detecting a level of binding of the peripheral benzodiazepine receptor ligand to the peripheral benzodiazepine receptor, relative to a level of binding in the absence of candidate agent, and therefrom identifying an agent that binds a peripheral benzodiazepine receptor. In one embodiment the sample comprises an intact cell that overexpresses a peripheral benzodiazepine receptor, and in certain further embodiments the cell is a permeabilized cell. In certain other further embodiments the cell is a neuronal cell. In another embodiment the peripheral benzodiazepine receptor is a mitochondrial peripheral benzodiazepine receptor. In another embodiment the peripheral benzodiazepine receptor ligand is detectably labeled, and in another embodiment the candidate agent is an agonist of the peripheral benzodiazepine receptor ligand. In another embodiment the candidate agent is an antagonist of the peripheral benzodiazepine receptor ligand. In certain other embodiments the peripheral benzodiazepine receptor ligand is PK-11195, 4-chlorodiazepam, DAA1106 or DAA1097.

In another embodiment the invention provides a method of screening for an agent that alters mitochondrial function, comprising the steps of (a) contacting, in the presence of a candidate agent (i) a sample comprising a mitochondrion from a cell that overexpresses a peripheral benzodiazepine receptor, and (ii) a peripheral benzodiazepine receptor ligand, and optionally (iii) a compound that alters mitochondrial membrane potential; (b) evaluating at least one mitochondrial function in the sample; and (c) comparing the mitochondrial function to a mitochondrial function detected in the absence of the candidate agent, and therefrom identifying an agent that alters mitochondrial function. In certain further embodiments the mitochondrial function is evaluated by determining mitochondrial membrane potential, and in certain other further embodiments the mitochondrial function is evaluated by detecting a level of apoptosis. In certain further embodiments the mitochondrion is present within an intact cell and in certain other further embodiments the mitochondrion is present within a permeabilized cell. In certain further embodiments the mitochondrion is present within a cell that overexpresses a peripheral benzodiazepine receptor and in certain other further embodiments the candidate agent is an agonist of the peripheral benzodiazepine receptor ligand. In certain further embodiments the candidate agent is an antagonist of the peripheral benzodiazepine receptor ligand, and in certain other further embodiments the peripheral benzodiazepine receptor ligand is PK-11195, 4-chlorodiazepam, DAA1106 or DAA1097. In certain embodiments the cell is a neuronal cell.

Turning to another embodiment, the present invention provides a method of screening for an agent that alters a mitochondrial function, comprising the steps of: (a) contacting, in the presence of a candidate agent (i) a cell that overexpresses a peripheral benzodiazepine receptor, (ii) a chemotherapeutic agent, and (iii) a peripheral benzodiazepine receptor ligand; (b) detecting a level of Bcl-2 binding to a Bcl-2 ligand in the cell; and (c) comparing the level of binding to a level of Bcl-2 binding to a Bcl-2 ligand detected in the absence of the candidate agent, and therefrom identifying an agent that alters a mitochondrial function. In certain further embodiments the cell overexpresses Bcl-2 and in other further embodiments the cell is a neuronal cell. In certain embodiments the cell is permeabilized. In certain embodiments the candidate agent is an agonist of the peripheral benzodiazepine receptor ligand and in certain embodiments the candidate agent is an antagonist of the peripheral benzodiazepine receptor ligand. In certain embodiments the peripheral benzodiazepine receptor ligand is PK-11195, 4-chlorodiazepam, DAA1106 or DAA1097. In certain embodiments the mitochondrial function is evaluated by determining mitochondrial membrane potential and in certain other embodiments the mitochondrial function is evaluated by detecting a level of apoptosis. In certain embodiments the step of comparing the level of apoptosis is by an assay determination that is vital dye staining of the cell, cell blebbing, caspase activity, DNA fragmentation, cytochrome c release or annexin binding to the cell. According to certain further embodiments the cell that overexpresses a peripheral benzodiazepine receptor is capable of being induced to express the peripheral benzodiazepine receptor.

In another aspect there is provided by the present invention a method for identifying a peripheral benzodiazepine receptor ligand that preferentially alters apoptosis, comprising (a) contacting, (i) a peripheral benzodiazepine receptor ligand, (ii) a cell that is capable of being induced to overexpress a peripheral benzodiazepine receptor, and (iii) an apoptogen, under conditions and for a time sufficient to induce apoptosis in said cell; and (b) comparing (i) a level of apoptosis in said cell that has been induced to overexpress a peripheral benzodiazepine receptor, to (ii) a level of apoptosis in said cell that has not been induced to overexpress a peripheral benzodiazepine receptor, wherein a decreased level of apoptosis in said cell that has been induced relative to the level of apoptosis in said cell that has not been induced indicates that the peripheral benzodiazepine receptor ligand preferentially alters apoptosis.

In another embodiment the invention provides a method for identifying a peripheral benzodiazepine receptor ligand that preferentially alters a mitochondrial function, comprising (a) contacting, (i) a peripheral benzodiazepine receptor ligand, (ii) a cell that is capable of being induced to overexpress a peripheral benzodiazepine receptor, and (iii) an agent that alters a mitochondrial function, under conditions and for a time sufficient to induce at least one altered mitochondrial function in said cell; and (b) comparing (i) a level of at least one mitochondrial function in said cell that has been induced to overexpress a peripheral benzodiazepine receptor, to (ii) a level of said at least one mitochondrial function in said cell that has not been induced to overexpress a peripheral benzodiazepine receptor, wherein a decreased level of the mitochondrial function in said cell that has been induced relative to the level of the mitochondrial function in said cell that has not been induced indicates that the peripheral benzodiazepine receptor ligand preferentially alters a mitochondrial function. In certain further embodiments the cell that is capable of being induced to overexpress a peripheral benzodiazepine receptor is of neuronal origin and the peripheral benzodiazepine receptor ligand is neuroprotective.

It is yet another aspect of the present invention to provide a cell line modified to express at least about ten-fold more peripheral benzodiazepine receptor protein than a parental cell line from which it is derived, and which overexpresses Bcl-2. In certain embodiments the cell line is modified to express at least about three-fold more Bcl-2 protein than a parental cell line from which it is derived. In certain other embodiments the parental cell line is a neuroblastoma cell line. In certain embodiments the cell line is designated S11. In another embodiment the invention provides a cell line modified to be capable of being induced to express at least about ten-fold more peripheral benzodiazepine receptor protein than a parental cell line from which it is derived, which in certain embodiments is modified to express at least about three-fold more Bcl-2 protein than a parental cell line from which it is derived. In certain other embodiments the parental cell line is a neuroblastoma cell line, and in certain embodiments the subject invention cell line is designated inducible PBzR overexpressing SH-SY5Y-derived cell line or IPBR-1.

These and other aspects of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1 is a histogram illustrating the detection of PBR ligand binding to PBR receptor in cell membranes. SY5Y neuroblastoma cells were transfected with a vector alone, comprising a gene encoding PBR or comprising the PBR gene in antisense orientation. Cell membrane protein was incubated with tritiated PK-11195, and the level of specific binding evaluated as cpm per 100 μg. Column 1 indicates the results for vector alone, column 2 shows the binding detected for vector encoding PBR, and column 3 presents the results for vector comprising PBR in the antisense orientation.

FIG. 2 is a histogram illustrating the detection of PBR ligand binding to PBR receptor in cell membranes. Jurkat cells were transfected with a vector alone or comprising a gene encoding human PBR. Cell membrane protein was incubated with tritiated PK-11195, and the level of specific binding evaluated as dpm per mg protein. Column 1 indicates the results for untransfected cells, column 2 shows the binding detected for vector encoding PBR, and column 3 presents the results for vector alone.

FIGS. 3A and 3B are graphs illustrating the saturation binding curves for tritiated PK-11195 in native (FIG. 3A) or PBR-transfected SY5Y cells. Specific binding (dpm) was evaluated at a series of levels of free PK-11195, as indicated.

FIG. 4 is a graph illustrating the saturation binding curve for tritiated PK-11195 in Jurkat cells. Specific binding (dpm) was evaluated at a series of levels of free PK-11195, as indicated.

FIG. 5 is a histogram comparing the specific binding of PK-11195 observed for a series of isolated SY5Y colonies stably transfected with PBR. Specific binding (dpm/10 μg protein) was evaluated for each colony).

FIG. 6 consists of three graphs illustrating PK-11195 binding. FIG. 6(A) shows PK-11195 saturation binding. FIG. 6(B) shows that at a fixed concentration of [³H]PK-11195, binding increased with increasing S11 protein. FIG. 6(C) shows that both RO 05-4864 (4-chlorodiazepam) and PK11195 displaced [³H]PK-11195.

FIG. 7 is a table of compounds screened in a PBzR ligand binding assay.

FIG. 8 is a western blot illustrating the binding of an antibody directed to the C-terminal peptide of PBzR. The subcellular fractions probed with the antibody are PNS, lysosome, and mitochondria.

FIG. 9 is a histogram illustrating specific PK-11195 binding to SY5Y neomycin resistant colonies.

FIG. 10 illustrates that over-expression of peripheral benzodiazepine receptor is localized to mitochondria of S11 cells. Four fractions were analyzed: homogenate, PNS, lysosomal, and mitochondrial.

FIG. 11 illustrates that acute PK-11195 treatment causes release of cytochrome C from S11 mitochondria upon calcium induced permeability transition.

FIG. 12 is a histogram illustrating that S11 cells are protected from PK-11195 induced cell death.

FIG. 13 is a histogram illustrating that S11 cells are more sensitive to etoposide induced caspase activation than vector controls.

FIG. 14 is a histogram illustrating the differential effects of ceramide and etoposide induced caspase activation in S11 and vector control cells.

FIG. 15 illustrates anti-VDAC antibody binding to S11 and vector control subcellular fractions: homogenate, PNS, lysosome, and mitochondria.

FIG. 16 illustrates that over-expression of PBzR in S11 cells correlates with increased Bcl-2 levels. FIG. 16(A) illustrates PBzR expression, and FIG. 16(B) illustrates Bcl-2 levels.

FIG. 17 illustrates that Bcl-2 levels, but not Bcl-XL levels, are increased in S11 mitochondria normalized to complex IV activity. FIG. 17(A), anti-Bcl-2; FIG. 17(B), anti-Bcl-XL.

FIG. 18 illustrates increased Bcl-2 levels in S11 cells and PBzR over-expression.

FIG. 19 shows expression of inducible PBzR in IPBR-1 inducible PBzR overexpressing SH-SY5Y-derived cells.

FIG. 20 shows the effect of induced PBzR overexpression on C₂-ceramide induced caspase activation in IPBR-1 cells.

FIG. 21 shows the effect of induced PBzR overexpression on doxorubicin induced caspase activation in IPBR-1 cells.

FIG. 22 shows the effects of induced PBzR overexpression on SIN-1 induced caspase activation and cell viability in IPBR1 cells.

FIG. 23 shows the protective effects of 4-chlorodiazepam against doxorubicin induced caspase activation in induced IPBR-1 cells.

### DETAILED DESCRIPTION OF THE INVENTION

As noted above, the present invention provides assays for use in identifying agents that alter mitochondrial function. Such assays are designed to detect an effect on binding of a PBR ligand to a mitochondrial peripheral benzodiazepine receptor (PBR). The present invention pertains in part to unexpected advantages provided by conducting such assays using biological samples derived from cells that overexpress PBR, and in particular, neuronal cells, hematopoietic cells and cells of other lineages. For example, PBR overexpression offers surprising sensitivity in certain screening assays that are rapid and that do not require excessive quantities of specific reagents. Thus, according to certain embodiments of the present invention there are provided assays, including high throughput screening assays, in which a sample comprising a cell that overexpresses a PBR is contacted with a PBR ligand and a candidate agent, and a level of PBR binding is detected. In certain other embodiments of the invention, PBR overexpression provides advantages in the context of assays for altered mitochondrial function as well.

A “biological sample” comprising a cell that overexpresses a PBR may comprise any tissue or cell preparation in which cells are present that have been genetically modified to express PBR at a level that is greater in a statistically significant manner relative to a control cell (e.g., the unmodified parental cell, a vehicle-only transfected control cell, a mock-transfected cell or the like) than the PBR expression level observed for the unmodified cell. Preferably a cell that overexpresses a PBR contains at least about two-fold more PBR protein than the unmodified cell from which it was derived, more preferably at least about five-fold more PBR protein and most preferably at least about ten-fold more PBR protein than the unmodified cell line from which it was derived. Overexpression may be achieved using any standard recombinant technique, using published PBR sequences (see, e.g., Carayon et al., 1996 Blood 87:3170). According to non-limiting theory, PBR overexpression provides a more sensitive assay read-out for screening candidate agents that may bind PBR and/or exert functional influences on one or more mitochondrial functions as provided herein.

Thus, for example, a biological sample may be a cell genetically modified to overexpress PBR that is derived from a normal (i.e., healthy) individual or from an individual having a disease associated with altered mitochondrial function, or a mitochondrion derived from such a cell. Biological samples may also be cells genetically modified to overexpress PBR, where such cells are derived by obtaining a blood sample, biopsy specimen, tissue explant, organ culture or any other tissue or cell preparation from a subject or a biological source, or a mitochondrion derived from such a cell. The subject or biological source may be a biological organism such as a human or non-human animal, a prokaryote or a eukaryote, a plant, a unicellular organism or a multicellular organism. The subject or biological source may also be a primary cell culture or culture adapted cell line including but not limited to genetically engineered cell lines that may contain chromosomally integrated or episomal recombinant nucleic acid sequences (including but not limited to a nucleic acid sequence responsible for PBR overexpression), immortalized or immortalizable cell lines, somatic cell hybrid or cytoplasmic hybrid “cybrid” cell lines (e.g., U.S. Pat. No. 5,888,498), differentiated or differentiatable cell lines, transformed cell lines and the like.

In certain embodiments, for example, a biological sample cell may be transfected with a gene encoding and expressing a biological receptor of interest, which may be a receptor having a known ligand (e.g., a cytokine, hormone or growth factor) or which may be an “orphaned” receptor for which no ligand is known. Further to such embodiments, one or more known ligands or other compounds suspected of being able to interact with the receptor of interest may be optionally contacted with the sample according to the subject invention method, for example, a cytokine, hormone, growth factor, antibody, neurotransmitter, receptor activator, receptor inhibitor, ion channel modulator, ion pump modulator, irritant, drug, toxin or any other compound known to have, or suspected of having, a biologically relevant activity.

According to certain embodiments contemplated by the present invention, a cell may be a permeabilized cell, which includes a cell that has been treated in a manner that results in loss of plasma membrane selective permeability. For example, it may be desirable to permeabilize a cell in a manner that permits calcium cations in the extracellular milieu to diffuse into the cell, as an alternative to the use of a calcium ionophore. As yet another example, certain candidate agents being tested according to the method of the present invention may not be able to pass through the plasma membrane, such that a permeabilized cell provides a suitable test cell for the potential effects of such agent. Those having ordinary skill in the art are familiar with methods for permeabilizing cells, for example by way of illustration and not limitation, through the use of surfactants, detergents, phospholipids, phospholipid binding proteins, enzymes, viral membrane fusion proteins and the like; through the use of osmotically active agents; by using chemical crosslinking agents; by physicochemical methods including electroporation and the like, or by other permeabilizing methodologies.

Thus, for instance, cells may be permeabilized using any of a variety of known techniques, such as exposure to one or more detergents (e.g., digitonin, Triton X-100™, NP-40™, octyl glucoside and the like) at concentrations below those used to lyse cells and solubilize membranes (i.e., below the critical micelle concentration). Certain common transfection reagents, such as DOTAP, may also be used. ATP can also be used to permeabilize intact cells, as may be low concentrations of chemicals commonly used as fixatives (e.g., formaldehyde). Accordingly, in certain embodiments of the invention, it may be preferred to use intact cells and in certain other embodiments the use of permeabilized cells may be preferred.

The term “screening” refers to the use of the invention to identify agents that alter (e.g., increase or decrease in a statistically significant manner relative to an appropriate control) binding of a PBR ligand to a PBR, or of a Bcl-2 ligand to Bcl-2, or that alter mitochondrial function, for instance, in a negative or positive fashion. Briefly, cells or portions thereof that comprise a mitochondrial PBR are treated with a candidate agent. The effect on PBR-ligand binding is then monitored and compared to a control sample that has been treated with only the vehicle used to deliver the agent. Detection may be direct (e.g., using a competitive binding assay) or indirect (e.g., based on an assay that detects mitochondrial function or Bcl-2 binding to a Bcl-2 ligand).

Direct Binding Assays

Certain assays provided herein are designed to directly monitor the effect of a candidate agent on binding of a PBR ligand to a PBR. Such assays are generally competitive binding assays, in which a PBR and PBR ligand are contacted, under conditions and for a time sufficient to permit detectable binding of PBR to PBR ligand. The assays is performed in the presence and absence of a candidate agent, and the effect of the candidate agent on binding of PBR ligand to PBR is evaluated. An agent that binds to PBR may result in a detectable decrease or increase in PBR ligand binding to PBR.

A PBR for use within the assays provided herein may be purified, or may be present within a sample. Preferably, the PBR is present within a mitochondrion, and more preferably within a mitochondrion-containing cell or fraction thereof (such as a membrane-containing fraction), and contact with the PBR ligand is achieved by incubating the cell in the presence of ligand. Preferred cells include, but are not limited to, neuronal cells, including primary cultures of neurons that have been modified to overexpress PBR and neuronal cell lines, for example the neuroblastoma cell line SH-SY5Y (ATCC, Manassas, Va.). Other preferred cells include hematopoietic cells that overexpress PBR, and in particular culture adapted hematopoietic cell lines excluding, however, the Jurkat human T lymphoblastoid cell line (Carayon et al., 1996 Blood 87:3170). Numerous other cells, cell types and cell lines that are well known may be used according to the present invention and it is particularly preferred that such cells overexpress PBR as provided herein. Suitable cells may also be, for example, cybrids (e.g., cytoplasmic hybrid cells comprising a common nuclear component but having mitochondria derived from different individuals). Methods for preparing and using cybrids are described in U.S. Pat. No. 5,888,438, published PCT applications WO 95/26973 and WO 98/17826, King and Attardi (Science 246:500-503, 1989), Chomyn et al. (Mol. Cell. Biol 11:2236-2244, 1991), Miller et al. (J. Neurochem. 67:1897-1907, 1996), Swerdlow et al. (Annals of Neurology 40:663-671, 1996), Cassarino et al. (Biochim. Biophys. Acta 1362:77-86, 1997), Swerdlow et al. (Neurology 49:918-925, 1997), Sheehan et al. (J. Neurochem. 68:1221-1233, 1997) and Sheehan et al. (J. Neurosci. 17:4612-4622, 1997).

Any PBR ligand may be used within such assays. A PBR ligand is any compound that binds detectably and specifically to a PBR using any standard binding assay. Whether a PBR ligand binds specifically to a PBR may be determined by determining the specific binding of the ligand, which is defined as the amount of a detectably labeled ligand that remains bound to a PBR in the presence of a 100-fold molar excess of unlabeled ligand subtracted from the amount of detectably labeled ligand that binds the PBR in the absence of unlabeled ligand, and which specific binding to the PBR will be greater in a statistically significant manner than the specific binding value determined for ligand binding to an irrelevant receptor. Preferably, a PBR ligand is readily detectable or may readily be detectably labeled, for example by covalent modification with one or more known labeling moieties There are a variety of compounds that are known PBR ligands including, for example, 4′chlorodiazepam (Ro 5-4864) and 1-(2-chlorophenyl)-N-methyl-N-(1-methylpropyl)-3-isoquinolinecarboxamide (PK11195). Other PBR ligands include N-(2,5-dimethoxy-benzyl)-N-(5-fluoro-2-phenoxyphenyl) acetamide (DAA1106; Funakoshi et al., 1999 Res. Commun. Mol. Pathol Pharmacol. 105:35-41; Chaki et al., 1999 Eur. Pharmacol. 371:197-204) and N-(4-chloro-2-phenoxyphenyl)-N-(2-isopropoxybenzyl) acetamide (DAA1097; Okuyama et al., 1999 Life Sci. 64:1455-64).

Preferably, the PBR ligand is labeled to facilitate detection of binding. Any suitable label may be employed, including radioactive groups, dyes, luminescent groups, fluorescent groups, biotin or an enzyme or substrate. Attachment of a label to a ligand may be achieved by any standard technique, and such techniques will be apparent to those having ordinary skill in the art. Contact of PBR with a PBR ligand may be achieved under any conditions that permit detectable binding in the absence of a candidate agent (e.g., in the absence of a potential inhibitor as known in the art and provided herein.

To evaluate the effect of a candidate agent, contact may be performed in the presence of candidate agent, and the resulting binding of PBR to PBR ligand compared to the level of binding in the absence of candidate agent. The candidate agent may be essentially any compound, such as a peptide, polynucleotide or small non-peptide molecule. Detection of binding may be by any suitable technique. Within embodiments in which the PBR ligand is labeled, binding may be assayed by detecting the amount of label associated with the PBR. The signal detected in the presence of candidate agent is compared to a reference signal obtained in the absence of candidate agent. An agent that results in a statistically significant alteration in the amount of label detected alters PBR association with ligand, and thus alters mitochondrial membrane permeability.

Assays Detecting Mitochondrial Function

Other assays provided herein identify agents that alter at least one mitochondrial function as provided herein, by evaluating the effect of a candidate agent on the ability of a known PBR ligand to influence mitochondrial function. Within such assays, a mitochondrion is contacted with a PBR ligand and a compound that influences at least one mitochondrial function, for example, a compound that alters mitochondrial membrane potential. Alternatively, for example, a chemotherapeutic agent, an apoptogen, an ionophore, a calcium cation, an uncoupler of oxidative phosphorylation from ATP production or any other other agent that directly or indirectly effects a change in a mitochondrial state may used in place of the compound that alters mitochondrial membrane potential. In the absence of candidate agent, the PBR ligand disrupts the mitochondrial membrane potential, leading to apoptosis. The effect of a candidate agent may be readily assayed by determining the effect on at least one mitochondrial function according to appropriate methodologies as known in the art and provided herein, for example, by determining membrane potential or by measuring apoptosis. Thus any such effect of altering (e.g., increasing or decreasing) mitochondrial function may be readily assayed using well known techniques.

Mitochondria for use within such assays may be isolated or present within PBR overexpressing cells which may, but need not, be cybrids as discussed above. For assays employing a chemotherapeutic agent, preferred cells also overexpress Bcl-2. Any PBR ligand as described herein may be employed in such assays.

Under certain conditions, a mitochondrial state which can feature altered mitochondrial regulation of intracellular calcium (e.g., altered mitochondrial membrane permeability to calcium) may be induced by exposing a biological sample to compositions referred to as “apoptogens” that induce programmed cell death, or “apoptosis”. A variety of apoptogens are known to those familiar with the art (see, e.g., Green et al., 1998 Science 281:1309 and references cited therein) and may include by way of illustration and not limitation: tumor necrosis factor-alpha (TNF-α); Fas ligand; glutamate; N-methyl-D-aspartate (NMDA); interleukin-3 (IL-3); herbimycin A (Mancini et al., 1997 J Cell. Biol. 138:449-469); paraquat (Costantini et al., 1995 Toxicology 99:1-2); ethylene glycols; protein kinase inhibitors, such as staurosporine, calphostin C, caffeic acid phenethyl ester, chelerythrine chloride, genistein; 1-(5-isoquinolinesulfonyl)-2-methylpiperazine; N-[2-((p-bromocinnamyl) amino)ethyl]-5-5-isoquinolinesulfonamide; KN-93; quercitin; d-erythro-sphingosine derivatives, for example, ceramide (e.g., C₂-ceramide); UV irradiation; ionophores such as ionomycin and valinomycin; MAP kinase inducers such as anisomycin, anandamine; cell cycle blockers such as aphidicolin, colcemid, 5-fluorouracil, homoharringtonine; acetylcholinesterase inhibitors such as berberine; anti-estrogens such as, tamoxifen; pro-oxidants, such as tert-butyl peroxide, hydrogen peroxide; free radicals such as nitric oxide; inorganic metal ions, such as cadmium; DNA synthesis inhibitors, including, for example, actinomycin D and also including DNA topoisomerase inhibitors, for example, etoposide; DNA intercalators such as doxorubicin, bleomycin sulfate, hydroxyurea, methotrexate, mitomycin C, camptothecin, daunorubicin; protein synthesis inhibitors such as cycloheximide, puromycin, rapamycin; agents that affect microtubulin formation or stability, for example, vinblastine, vincristine, colchicine, 4-hydroxyphenylretinamide, paclitaxel; Bad protein, Bid protein and Bax protein (see, e.g., Jurgenmeier et al., 1998 Proc. Nat. Acad. Sci. USA 95:4997-5002 and references cited therein); calcium and inorganic phosphate (Kroemer et al., 1998 Ann. Rev. Physiol. 60:619).

Following contact of the assay components, a mitochondrial function is evaluated. Preferably, the mitochondrial function is evaluated by assaying mitochondrial membrane potential or apoptosis. Mitochondrial membrane potential may be determined according to methods familiar to those skilled in the art, including but not limited to detection and/or measurement of indicator compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals-Sixth Ed., Molecular Probes, Eugene, Oreg., pp. 266-274 and 589-594.). Many such indicators are known in the art, and suitable indicators include the fluorescent probes 2-,4-dimethylaminostyryl-N-methyl pyridinium (DASPMI), tetramethylrhodamine esters (such as, e.g., tetramethylrhodamine methyl ester, TMRM; tetramethylrhodamine ethyl ester, TMRE) and related compounds (see, e.g., Haugland, 1996, supra). Such probes may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, at http://www.probes.com/ handbook/toc.html). Other fluorescent indicator compounds that may be used include, but are not limited to, rhodamine 123, rhodamine B hexyl ester, DiOC₆(3), JC-1 [5,5′, 6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Göttingen, Germany; see Sakanoue et al., 1997 J. Biochem. 121:29).

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J Physiol. 269:R1213). Those skilled in the art will be able to select appropriate indicator compounds or other appropriate means for measuring ΔΨm.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorometric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.) Alternatively, mitochondrial membrane potential may be measured using a method described in co-pending application entitled “Compositions and Methods for Assaying Subcellular Conditions and Processes using Energy Transfer” (U.S. Provisional Application No. 60/140,433). By “capable of maintaining a potential” it is meant that such mitochondria have a membrane potential that is sufficient to permit the accumulation of a detectable, potential-sensitive or potentiometric compound, for example, the fluorescent dyes rhodamine 123, DASPMI [2-,4-dimethylaminostyryl-N-methylpyridinium], TMRM [tetramethyl rhodamine methyl ester] or other suitable compounds (see, e.g., Scheffler, Mitochondria, 1999 Wiley-Liss, NY, pp. 198-202; see also Haugland, 1996).

Alternatively, any of a variety of apoptosis assays may be used. For example, apoptosis in many cell types causes an altered morphological appearance such as plasma membrane blebbing, cell shape change, loss of substrate adhesion properties or other morphological changes that can be readily detected by those skilled in the art using light microscopy. As another example, cells undergoing apoptosis may exhibit fragmentation and disintegration of chromosomes, which may be apparent by microscopy and/or through the use of DNA specific or chromatin specific dyes that are known in the art, including fluorescent dyes. Such cells may also exhibit altered membrane permeability properties as may be readily detected through the use of vital dyes (e.g., propidium iodide, trypan blue) or the detection of lactate dehydrogenase leakage into the extracellular milieu. Damage to DNA may also be assayed using electrophoretic techniques (see, for example, Morris et al., BioTechniques 26:282-289, 1999). These and other means for detecting apoptotic cells by morphologic, permeability and related changes will be apparent to those familiar with the art.

In another apoptosis assay, translocation of cell membrane phosphatidylserine (PS) from the inner to the outer leaflet of the plasma membrane may be quantified by measuring outer leaflet binding by the PS-specific protein annexin (Martin et al, J. Exp. Med. 182:1545-1556, 1995; Fadok et al., J. Immunol. 148:2207-2216, 1992.). In a preferred format, exteriorization of plasma membrane PS is assessed in 96-well plates using a labeled annexin derivative such as an annexin-fluorescein isothiocyanate conjugate (annexin-FITC, Oncogene Research Products, Cambridge, Mass.).

In another apoptosis assay, quantification of the mitochondrial protein cytochrome c that has leaked out of mitochondria in apoptotic cells may provide an apoptosis indicator that can be readily determined (Liu et al., Cell 86:147-157, 1996). Such quantification of cytochrome c may be performed spectrophotometrically, immunochemically or by other well established methods for detecting the presence of a specific protein. Release of cytochrome c from mitochondria in cells challenged with apoptotic stimuli (e.g., ionomycin, a well known calcium ionophore) can be followed by a variety of immunological methods. Matrix-assisted laser desorption ionization time of flight mass (MALDI-TOF) spectrometry coupled with affinity capture is particularly suitable for such analysis since apo-cytochrome c and holo cytochrome c can be distinguished on the basis of their unique molecular weights. For example, the SELDI system (Ciphergen, Palo Alto, USA) may be utilized to follow the inhibition by mitochondria protecting agents of cytochrome c release from mitochondria in ionomycin treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular weight of the protein is determined by its time of flight to the detector of the SELDI mass spectrometer.

In another apoptosis assay, induction of specific protease activity in a family of apoptosis-activated proteases known as the caspases (Thornberry and Lazebnik, Science 281:1312-1316, 1998) is measured, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., J. Neurosci. 17:6165-6178, 1997). The labeled synthetic peptide Z-Tyr-Val-Ala-Asp-AFC, wherein “Z” indicates a benzoyl carbonyl moiety and AFC indicates 7-amino-4-trifluoromethylcoumarin (Kluck et al., 1997 Science 275:1132-1136, 1997; Nicholson et al., Nature 376:37-43, 1995), is one such substrate. Another labeled synthetic peptide substrate for caspase-3 consists of two fluorescent proteins linked to each other via a peptide linker comprising the recognition/cleavage site for the protease (Xu et al., Nucleic Acids Res. 26:2034-2035, 1998). Other substrates include nuclear proteins such as U1-70 kDa and DNA-PKcs (Rosen and Casciola-Rosen, J. Cell. Biochem. 64:50-454, 1997; Cohen, Biochem. J. 326:1-16, 1997).

In yet another apoptosis assay, the ratio of living to dead cells, or the proportion of dead cells, in a population of cells may be determined as a measure of the ultimate consequence of apoptosis. Living cells can be distinguished from dead cells using any of a number of techniques known to those skilled in the art. By way of non-limiting example, vital dyes such as propidium iodide or trypan blue may be used to determine the proportion of dead cells in a population of cells that have been treated with an apoptogen and a compound according to the invention.

The person of ordinary skill in the art will readily appreciate that there may be other suitable techniques for quantifying apoptosis, and the use of any such techniques for purposes of determining the effects of agents on the induction and kinetics of apoptosis are within the scope of the assays disclosed here.

According to certain embodiments of the present invention there are provided methods for characterizing a PBR ligand according to the ability of such a ligand to preferentially alter a mitochondrial function as provided herein, which in certain preferred embodiments includes the ability of such a ligand to preferentially alter apoptosis. Without wishing to be bound by theory, in these and related embodiments it is believed that not all PBR ligands interact with the PBR in precisely the same way, and that at least some PBR ligands may also interact with intracellular components other than the PBR. For example, the structure of the PBR is believed to provide a number of distinct exposed sites for intermolecular interactions, such that various PBR ligands may bind to or directly or indirectly influence the PBR at different sites (see, e.g., Liauzun et al., 1999 J. Biol. Chem. 273:2146). Similarly, whether conditions permit a given PBR ligand that is capable of interacting with PBR as well as with other intracellular components to in fact interact with PBR may depend in part on the quantitative presence (e.g., availability) of PBR. Thus, a PBR ligand that “preferentially” alters a mitochondrial function or that “preferentially” alters apoptosis refers to a ligand that differentially induces such alteration (e.g., a statistically significant increase or decrease in at least one mitochondrial function or in apoptosis) in a cell that is capable of being induced to overexpress a PBR and that has been so induced to overexpress PBR, relative to such a cell in an uninduced state with respect to PBR overexpression.

By way of background, spontaneous PBR overexpression has been detected in a number of primary tumors (e.g., glioma, Black et al Cancer 1990 65:93-97; hepatocellular carcinoma, Venturini, et al 1998 Life Science 63:1269-1280; breast cancer, Hardwick et al 1999 Cancer Res. 59:831-842; lymphoma, Laird et al 1989 Eur. J. Pharmacol. 171: 25-35; ovarian cancer, Batra, et a,l 1998 Int. J Oncol. 1998 12:1295-8; astrocytoma, Miettinen et al 1995 Cancer Res. 12:2691-2695). The present invention contemplates the non-limiting possibility that such PBR overexpression associated with malignancy and/or metastatic potential may underlie resistance of such tumors to certain chemotherapeutic agents, and provides the surprising discovery that inducible PBR overexpression permits distinctions to be made among PBR ligands, as provided herein and described further in the Examples. The present invention thus provides an opportunity to distinguish among the mechanisms of action of different chemotherapeutic agents (including those which may be PBR ligands) by determining the relative importance of PBR in such mechanisms, i.e., by providing a method to identify preferential alteration of a mitochondrial function (e.g., apoptosis). The invention thus relates PBR ligand efficacy to mitochondrial function, e.g., apoptosis, where a refractory state to a chemotherapeutic agent (e.g., an apoptogen) may be a property of cancer cells that is usefully predicted and overcome through selection of a suitable PBR ligand according to the subject invention methods and compositions.

Assays Detecting Bcl-2 Interactions

The bcl-2 gene was initially identified as a causal factor in certain types of lymphatic cancers (B-cell lymphoma) in which bcl-2 is overexpressed, resulting in an abnormally longer lifespan for B-cells. This longer lifespan appears to allow these cells to accumulate additional mutations resulting in frank malignancy and lymphatic tumor development (for reviews of the Bcl-2 family of proteins, see Davies, Trends in Neuroscience 18:355-358, 1995; Kroemer, Nature Med. 3:614-620, 1997; WO95/13292; WO95/00160; and U.S. Pat. No. 5,015,568). Although the biochemical function of Bcl-2 is not known (i.e., it is not clear whether it acts as an enzyme, receptor or signaling molecule), it is known to be localized to the outer mitochondrial membrane, the nuclear membrane and the endoplasmic reticulum.

It has been found, within the context of the present invention, that in certain cell lines (e.g., SH-SY5Y-derived PBR-overexpressing clone S11 as described in greater detail below), transfection with a PBR gene is accompanied by an increase in the level of Bcl-2 expression. Further, Bcl-2 overexpression in model cancer cell lines (e.g., Jurkat and human T cell lymphoma cell lines) has been found to protect the cells from entering apoptosis when treated with an apoptogenic agent. The protective effect of Bcl-2 can be overcome by exposing the cells to a chemotherapeutic agent in combination with a PBR ligand, where neither the chemotherapeutic agent nor the PBR ligand itself overcomes the Bcl-2 effect. Accordingly, assays to screen for agents that modulate Bcl-2 binding to a Bcl-2 ligand may be used to identify agents that alter at least one mitochondrial function, for example, mitochondrial membrane potential.

Within such screens, for example, a cell that comprises a mitochondrion and overexpresses a PBR is contacted with a chemotherapeutic agent and a PBR ligand. The level of Bcl-2 binding to a Bcl-2 ligand in the cell is then assayed. The level of such binding in the presence of a candidate agent is compared to the level of binding in the absence of candidate agent. Agents that that alter the interaction of Bcl-2 and ligand generally alter mitochondrial membrane potential. Suitable cells to be used as samples are described above; preferred cells overexpress Bcl-2. PBR ligands are generally as described above. The chemotherapeutic agent may be any agent that induces cell death and is preferably an agent that induces apoptosis. Examples of chemotherapeutic agents include the anti-neoplastic agents lonidamine, cisplatin, doxorubicin, cyclophosphamide and may also include apoptogens as provided herein.

Therapeutic Applications

Agents identified using the above assays may have remedial, therapeutic, palliative, rehabilitative, preventative and/or prophylactic effects on patients suffering from, or potentially predisposed to developing, diseases and disorders associated with alterations in mitochondrial function. Such diseases may be characterized by abnormal, supernormal, inefficient, ineffective or deleterious activity, for example, defects in uptake, release, activity, sequestration, transport, metabolism, catabolism, synthesis, storage or processing of biological molecules and macromolecules such as proteins and peptides and their derivatives, carbohydrates and oligosaccharides and their derivatives including glycoconjugates such as glycoproteins and glycolipids, lipids, nucleic acids and cofactors including ions, mediators, precursors, catabolites and the like.

Such diseases and disorders include, by way of example and not limitation, chronic neurodegenerative disorders such as Alzheimer's disease (AD) and Parkinson's disease (PD); auto-immune diseases; diabetes mellitus, including Type I and Type II; mitochondria associated diseases, including but not limited to congenital muscular dystrophy with mitochondrial structural abnormalities, fatal infantile myopathy with severe mtDNA depletion and benign “later-onset” myopathy with moderate reduction in mtDNA, MELAS (mitochondrial encephalopathy, lactic acidosis, and stroke) and MIDD (mitochondrial diabetes and deafness); MERFF (myoclonic epilepsy ragged red fiber syndrome); arthritis; NARP (Neuropathy; Ataxia; Retinitis Pigmentosa); MNGIE (Myopathy and external ophthalmoplegia; Neuropathy; Gastro-Intestinal; Encephalopathy), LHON (Leber's Hereditary Optic Neuropathy), Kearns-Sayre disease; Pearson's Syndrome; PEO (Progressive External Ophthalmoplegia); Wolfram syndrome; DIDMOAD (Diabetes Insipidus, Diabetes Mellitus, Optic Atrophy, Deafness); Leigh's Syndrome; dystonia; schizophrenia; and hyperproliferative disorders, such as cancer, tumors and psoriasis.

Agents administered for therapeutic purposes are preferably formulated within a pharmaceutical composition. Pharmaceutical compositions comprise one or more such agents in combination with a physiologically acceptable carrier or excipient. Such compositions may be in the form of a solid, liquid or gas (aerosol). Alternatively, compositions of the present invention may be formulated as a lyophilizate. Agents may also be encapsulated within liposomes using well known technology. Pharmaceutical compositions within the scope of the present invention may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

For peptide or protein agents, a pharmaceutical composition may alternatively contain a polynucleotide encoding the agent, such that the agent is generated in situ. Within such compositions, the DNA may be present within any of a variety of delivery systems known to those of ordinary skill in the art, including nucleic acid expression systems, bacterial and viral expression systems and mammalian expression systems. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Any suitable carrier known to those of ordinary skill in the art may be employed in the pharmaceutical compositions described herein. Carriers and excipients for therapeutic use are well known, and are described, for example, in Remingtons Pharmaceutical Sciences, Mack Publishing Co. (A. R. Gennaro ed. 1985). In general, the type of carrier is selected based on the mode of administration. Compositions of the present invention may be formulated for any appropriate manner of administration, including for example, topical, oral, nasal, intrathecal, rectal, vaginal, sublingual or parenteral administration, including subcutaneous, intravenous, intramuscular, intrasternal, intracavernous, intrameatal or intraurethral injection or infusion. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A composition (e.g., for oral administration or delivery by injection) may be in the form of a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. When intended for oral administration, preferred compositions contain, in addition to one or more agents that alter ΔΨm, one or more of a sweetening agent, preservatives, dye/colorant or flavor enhancer. In a composition intended to be administered by injection, one or more of a surfactant, preservative, wetting agent, dispersing agent, suspending agent, buffer, stabilizer or isotonic agent may be included. The use of physiological saline is preferred, and an injectable pharmaceutical composition is preferably sterile.

A liquid composition intended for either parenteral or oral administration should contain an amount of agent that affects ΔΨm such that a suitable dosage will be obtained (e.g., at least 0.01 wt % of agent). When intended for oral administration, this amount may be varied to be between 0.1 and about 70% of the weight of the composition. Preferred oral compositions contain between about 4% and about 50% of such agent(s). Preferred compositions and preparations are prepared so that a parenteral dosage unit contains between 0.01 to 1% by weight of active compound.

For certain topical applications, formulation as a cream or lotion, using well known components, is preferred. For example, a carrier may be a solution, emulsion, ointment or gel base comprising, for example, one or more of petrolatum, lanolin, a polyethylene glycol, beeswax, mineral oil, a diluent (such as water or alcohol), an emulsifier or a stabilizer. Thickening agents may also be present in a pharmaceutical composition for topical administration. If intended for transdermal administration, a composition may be present within a transdermal patch or iontophoresis device. Topical formulations may contain a concentration of the agent that affects ΔΨm of from about 0.1 to about 10% w/v (weight per unit volume).

The composition may be intended for rectal administration (e.g., in the form of a suppository which will melt in the rectum and release the drug). A composition for rectal administration may contain an oleaginous base as a suitable nonirritating excipient. Such bases include, for example, lanolin, cocoa butter and polyethylene glycol.

The compositions described herein may be formulated for sustained release (i.e., a formulation such as a capsule or sponge that effects a slow release of compound following administration). Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Within a pharmaceutical composition, an agent that affects ΔΨm may be linked to any of a variety of compounds. For example, such an agent may be linked to a targeting moiety (e.g. a monoclonal or polyclonal antibody, a protein or a liposome) that facilitates the delivery of the agent to the target site. As used herein, a “targeting moiety” may be any substance (such as a compound or cell) that, when linked to an agent enhances the transport of the agent to a target cell or tissue, thereby increasing the local concentration of the agent. Targeting moieties include antibodies or fragments thereof, receptors, ligands and other molecules that bind to cells of, or in the vicinity of, the target tissue. Known targeting moieties include, for example, serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. An antibody targeting agent may be an intact (whole) molecule, a fragment thereof, or a functional equivalent thereof. Examples of antibody fragments are F(ab′)2,-Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) at which the agent is expected to exert a therapeutic benefit.

Pharmaceutical compositions may be administered in a manner appropriate to the disease to be treated (or prevented). Appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an amount sufficient to provide therapeutic and/or prophylactic benefit (e.g., an improved clinical outcome, such as more frequent complete or partial remissions, or longer disease-free and/or overall survival). Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Species-specific Agents

In certain embodiments, the present invention provides screening assays for identifying species-specific agents. A “species-specific agent” refers to an agent that alters mitochondrial function, for example mitochondrial membrane potential of one source (e.g., species) but that does not substantially affect the mitochondrial membrane potential of a second source. Typically, the agent should have an effect on one species that is at least twice the effect on the other species. The screening assays provided herein may be used to identify such agents, using cells and/or mitochondria obtained from different biological sources.

This embodiment of the invention may be used, for example, to identify agents that selectively induce collapse of Δψ in mitochondria derived from different species, e.g., in trypanosomes (Ashkenazi et al., Science 281:1305-1308, 1998), and other eukaryotic pathogens and parasites, including but not limited to insects, but which do not induce Δψ collapse in the mitochondria found in the cells of their mammalian hosts. Such agents are expected to be useful for the prophylactic or therapeutic management of such pathogens and parasites.

By way of another example, members of the phylum Apicomplexa (formerly called Sporozoa) comprise a large and diverse group of pathogenic protozoa that are intracellular parasites. Some members, including species of Babesia, Theileria and Eimeria, cause economically important animal diseases, and other members, such as Toxoplasma gondii and Cryptosporidium spp. also cause human disease, particularly in immunocompromised individuals. The acomplexicans are unusual in terms of their extrachromosomal DNA elements, as they comprise both a mitochondrial genome and a putative plastid genome (see Feagin, Annu. Rev. Microbiol. 48:81-104, 1994, for a review). Probably the most well-studied acomplexicans are species of Plasmodium, which cause malaria. Antimalarial agents include agents that specifically impact the function of Plasmodium mitochondria (Peters et al., Ann. Trop. Med. Parsitol. 78:567-579, 1984; Basco et al., J. Eukaryot. Microbiol. 41:179-183, 1994), and one such agent, atovaquone, collapses Δψ in mitochondria from Plasmodium yoelii but has no effect on Δψ of mammalian mitochondria (Srivastava et al., J. Biol. Chem. 272:3961-3966, 1997). Accordingly, the assays provided herein can be used to screen libraries of compounds for novel antimalarial agents, such as compounds that cause Δψ collapse in Plasmodium mitochondria but not in mammalian mitochondria.

As another example, screening methods provided herein may be used to identify agents that selectively induce Δψ collapse in mitochondria derived from undesirable plants (e.g., weeds) but not in desirable plants (e.g., crops), or in undesirable insects (in particular, members of the family Lepidoptera and other crop-damaging insects) but not in desirable insects (e.g., bees) or desirable plants. Such agents are expected to be useful for the management and control of such undesirable plants and insects. Cultured insect cells, including for example, the Sf9 and Sf21 cell lines derived from Spodoptera frugiperda, and the HIGH FIVE™ cell line from Trichopolusia ni (these three cell lines are available from Invitrogen, Carlsbad, Calif.) may be the source of mitochondria in certain such embodiments of the invention.

The following Examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### EXAMPLES

### Example 1

Binding of PBR Ligand to PBR

This Example illustrates the detection of PBR ligand binding to a PBR in cell membranes.

Standard molecular biology reagents and methodologies were used as described, for example, in Ausubel et al. (Current Protocols in Molecular Biology, Greene Publishing, 1987); and in Sambrook et al. (Molecular Cloning: A Laboratory Manual, Cold Spring Harbor Press, 1989). All reagents were from Sigma (St. Louis, Mo.) unless otherwise specified. Full length PBzR cDNA (peripheral benzodiazepine receptor, GENBANK Accession No. NM_{—}000714; 1991 Eur. J. Biochem. 195:305-311; see, e.g., Carayon et al., 1996 Blood 87:3170 was amplified from human placental cDNA and cloned into pcDNA3.1 (Invitrogen, Carlsbad, Calif.) following the supplier's recommendations, in sense and antisense orientations. SY5Y neuroblastoma cells and Jurkat T lymphoblastoid cells (ATCC, Manassas, Va.) were each transfected with a vector comprising the PBR gene in sense orientation and, separately, with the PBR antisense construct. Stable colonies were selected using G418 and isolated according to standard cell culture techniques, and cell membranes were prepared by harvesting cells in PBS containing 5 mM EDTA, pelleting the cells by centrifugation and resuspending the cell pellet in binding buffer (25 mM Tris, 10 mM MgCl₂, pH 7.5). Aliquots of the cell membrane preparation containing 38 μg membrane protein were incubated in binding buffer supplemented with with 100 μM tritiated PK-11195 (86 Ci/mmol; New England Nuclear, Boston, Mass.). The mixture was incubated on ice for 60 minutes, and unbound ligand was removed by GF/C glass fiber filtration.

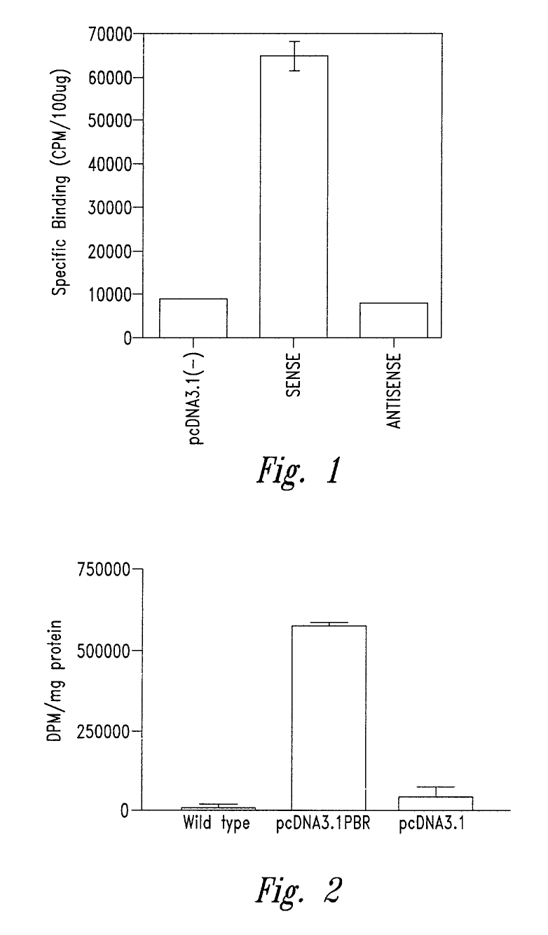

The results, presented in FIG. 1, show that the PBR ligand specifically binds the cell membranes. The result obtained for vector alone (i.e., lacking a PBR sequence) is similar to the level of binding observed in native, untransformed cells. Results for a similar experiment, using transfected Jurkat cells (ATCC) and 6 nM PK-11195 are presented in FIG. 2.

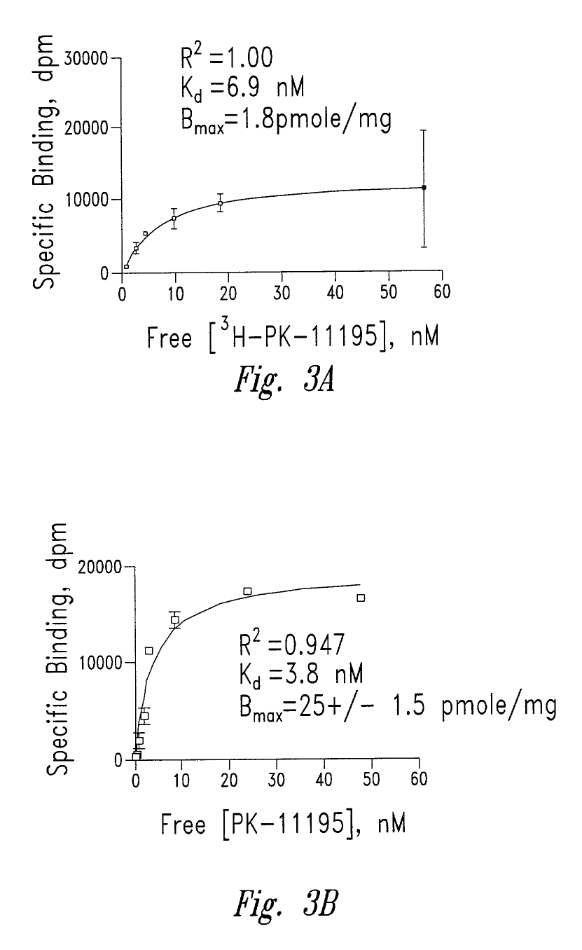

Saturation binding curves obtained for native and stably PBR-transfected SY5Y cells are presented in FIGS. 3A and 3B, respectively. These curves indicate that the K_{d} for native cells (6.9 nM) is similar to the K_{d} for transfected cells (3.8 nM). The binding maximum (pmol/mg, B_{MAX}) is approximately 10 fold higher in stable PBR expressing cells (25±1.5 pmol/mg) than in native cells (1.8 pmol/mg).

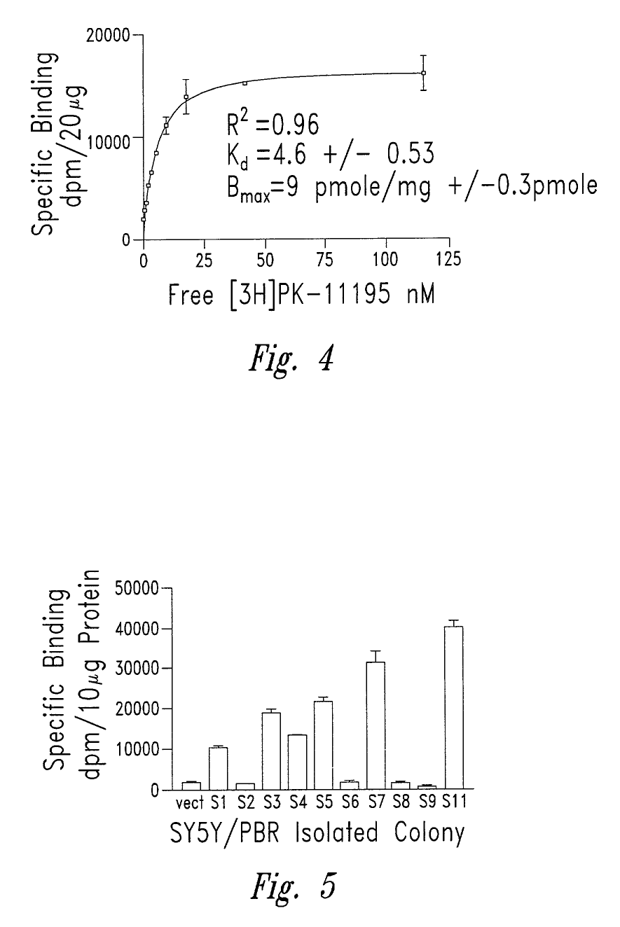

A saturation binding curve was also obtained for Jurkat cells stably expressing PBR (FIG. 4). Each assay point contained 20 μg of membrane protein, which yielded readily detectable signal with little ligand depletion. 100 μM RO-05-4864 (4-chlorodiazepam) was used for non-specific binding. At 6 nM PK-11195, the signal:noise ratio (total binding:non-specific binding) was about 4:1. The curve fit a one-site binding model and had a K_{d} of about 3 nM, indicating correct receptor folding. Bₘₐₓ was about 9 pmol/mg protein.

PK-11195 binding was also evaluated for a series of isolated SY5Y colonies stably transfected with PBR. Binding assays were performed at 6 nM tritiated PK-11195 and 100 μM RO-05-4864 (4-chlorodiazepam) for non-specific binding. Each assay contained 10 μg of membrane protein. The colonies displayed a range of specific binding, up to 20 fold greater specific binding in the case of clone S11 (FIG. 5). Background counts did not vary significantly from clone to clone and the signal:noise was about 20:1 for S11, 5:1 for stable colonies described above and 2:1 for native SY5Y cells.

### Example 2

Characterization of PBzR Binding

To demonstrate expression of PBzR, neomycin resistant cells were characterized by a radioligand binding assay using radiolabeled PK-11195. PK-11195 is an isoquinoline which is specific for PBzR and does not interact with the CNS GABA channel or central benzodiazepine receptor (Le Fur et al., Life Sciences 33:449-457, 1983). Initially, resistant cells were analyzed in ligand binding experiments. The ligand binding assays were performed by harvesting cells with PBS containing 5 mM EDTA and resuspending them in ice cold 25 mM Tris pH 7.5, 10 mM MgCl₂ so that the cells no longer exclude trypan blue. Bound ligand is separated from free ligand using GF/C glass fiber filters. Table 1 displays the K_{d} and Bₘₐₓ values obtained from saturation binding curves analyzed by non-linear regression in pooled SY5Y cells and Jurkat cells.

TABLE 1

| Cell Line | K_{d} (nM) | Bₘₐₓ (pmol/mg) |
|---|---|---|
| SH-SY5Y Vector | 6.9 | 1.8 |
| S11 | 5.3 | 43 |
| Jurkat Vector | Not detected | Not detected |
| Jurkat PBzR | 4.6 | 9 |

The Kd's from the over-expressing cell lines are very similar to the native SH-SY5Y cell line and the reported value of 2-4 nM indicates that the over-expressed receptor is folding properly (Le Fur et al., 1983). In each cell line, PBzR is dramatically over-expressed relative to the native cell line, 10 fold in the case of pooled PBzR over-expressing SH-SY5Y cells. The data from this experiment also show that the signal to noise ratio in the native SY5Y cell line is 2:1 vs. 7:1 in the pooled PBzR over-expressing SY5Y cell line. The pooled PBzR over-expressing Jurkat line is 4:1. Thus, the over-expressing cell lines provide considerably more sensitivity for screening compound libraries than the native cell lines. The improved signal to noise ratio is preferred for displacement assays, such as high throughput displacement assays, as the typical working concentration of PK-11195 (0.3-0.5 nM) yields a virtually undetectable signal in native cell lines.

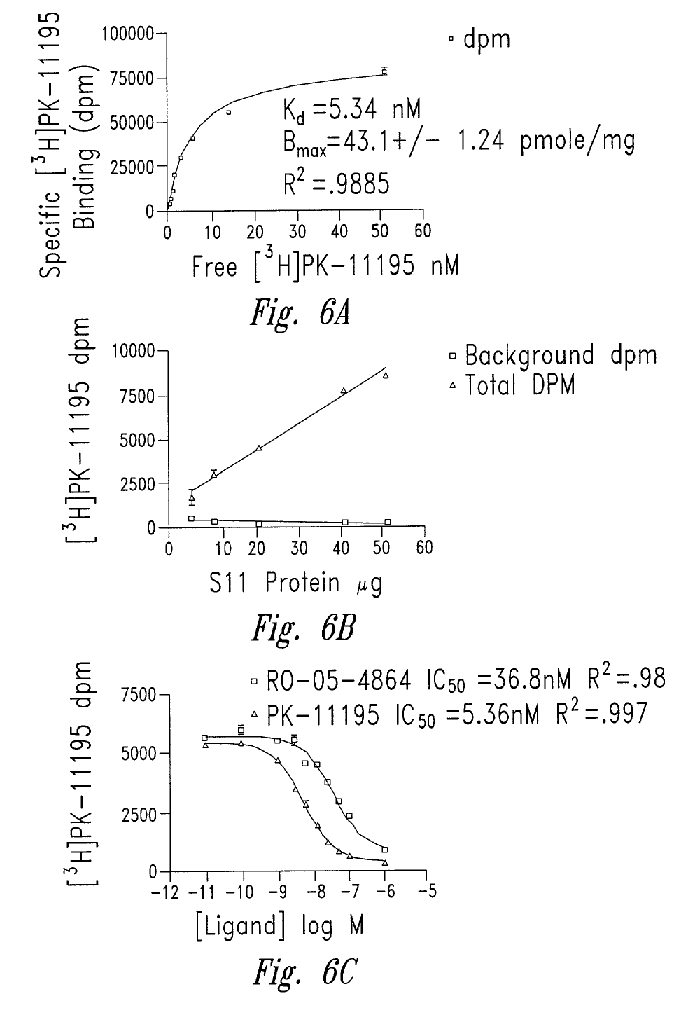

Additional experiments were performed to further characterize the binding of ligand to PBzR. The specificity of [³H]PK-11195 binding was examined by testing the ability of PK-11195 and RO 05-4864 (4-chlorodiazepam), known pBdz ligands, to displace [³H]PK-11195 binding to the S11 PBzR overexpressing clone, which is described in Example 3. The results are shown in FIG. 6. FIG. 6(A) illustrates PK-11195 saturation binding using 10 μg S11 protein per data point. FIG. 6(B) illustrates that at a fixed concentration (0.5 nM), [³H]PK-11195 binding increased with increasing S11 protein, from 0-25 μg. As shown in FIG. 6(C), both RO 05-4864 and PK-11195 displaced [³H]PK-11195 using 6 μg S11 protein per data point; the IC₅₀ value for PK-11195 was 5.36 nM R² and the IC₅₀ value for RO 05-4864 was 36.8 nM R².

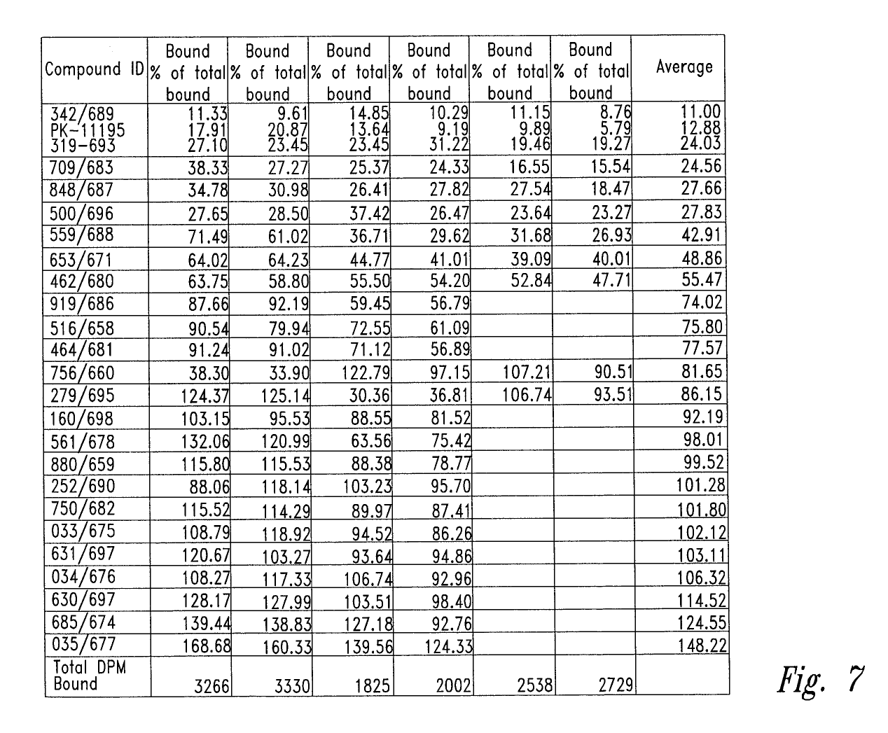

Compounds from a compound library were screened in the PBzR ligand binding assay. The results are shown in FIG. 7.

### Example 3

Mitochondrial Localization of PBzR in S11 Cells and Effect on Mitochondrial Function

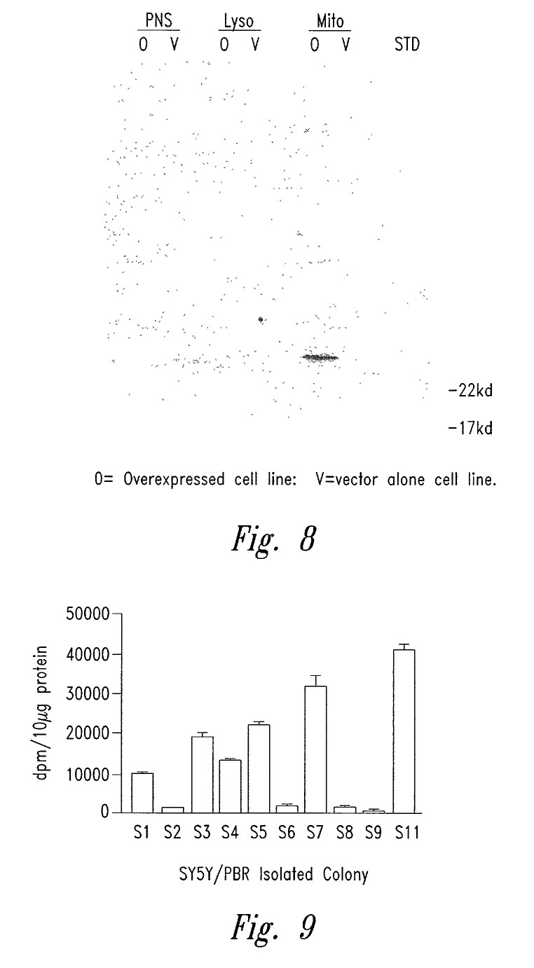

Since PBzR is normally localized to the mitochondria, it was important to determine whether PBzR overexpression is mitochondrial or ectopic. PBzR subcellular localization was determined by fractionating subcellular organelles from SY5Y cells overexpressing PBzR (or from control cells transfected with the empty vector) using metrizamide gradients as described by Storrie and Madden (1990 Meths. Enzymol. 182:203-225). Three membrane fractions were isolated and found to be enriched for activities known to be localized to the post-nuclear supernatant (PNS), lysosomes and mitochondria. The mitochondrial fraction was enriched ten-fold in cytochrome C oxidase activity and at least five-fold in specific PK-11195 binding. Western blots of fractions were probed using a mitochondrial ETC complex IV-reactive antibody, which demonstrated some mitochondrial contamination in all fractions but a 5-7-fold enrichment in the mitochondrial fraction. A polyclonal antibody was developed that was directed to the C-terminal peptide of PBzR (other antibodies to PBzR are commercially available, e.g., from Biovision, Inc., Palo Alto, Calif. and R&D Systems, Minneapolis, Minn.). This antibody was used to probe western blots of the above fractions as shown in FIG. 8.

According to the results shown in FIG. 8, the antibody reacted exclusively with an 18 kD protein found in the mitochondrial fraction of pooled, selected (e.g., oligoclonal) PBzR over-expressing cells but not the vector control. Although native SY5Y cells showed detectable PK-11195 binding, the receptor concentration was ten fold higher in the over-expressing cells, accounting for the apparent lack of detectable signal from the mitochondrial fraction of empty vector-transfected control cells.

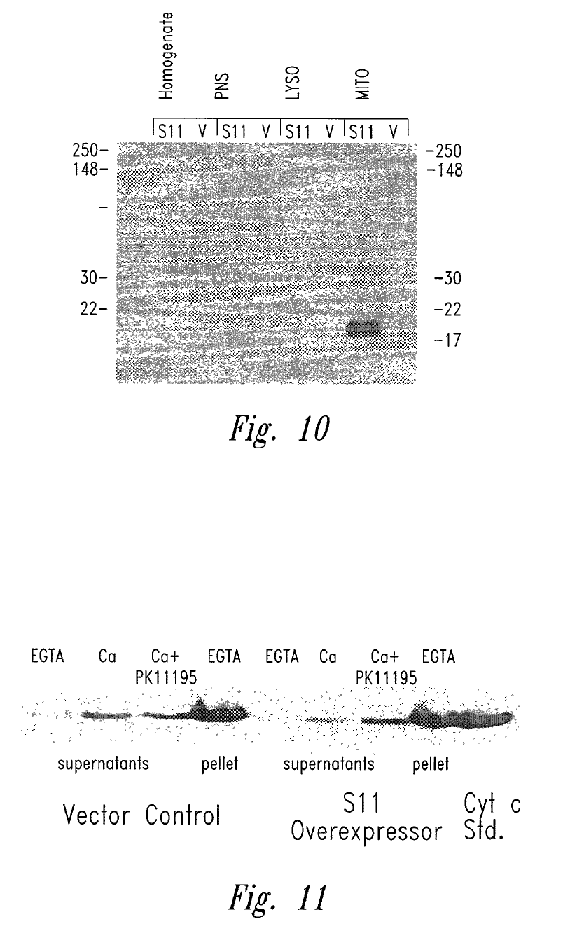

To isolate a clonal cell line, SY5Y neomycin resistant colonies were isolated using cloning rings and expanded from twelve well plates. FIG. 9 shows the range of specific PK-11195 binding in the isolated cell lines S1, S2, S3, S4, S5, S6, S7, S8, S9, and S11 at 6 nM concentration of [³H]PK-11195. Binding was highest for cell line S11 .

As described in Example 1, increases in PBzR expresison levels, as evidenced by specific binding of [³H]PK-11195, ranged in value from essentially no overexpression to at least about twenty-fold overexpression in the case of S11, in assays using 6 nM [³H]PK-11195. Saturation binding curves using the S11 SY5Y clone indicated a receptor concentration of 25 pMol/mg in the pooled over-expressors. Displacement binding curves with a fixed concentration of [³H]PK-11195 at 0.35 nM were performed using unlabeled PK-11195 and RO-05-4864. The S11 cell line was used as a source of PBzR. An example of this curve is shown in FIG. 6(C).

Overexpression of the peripheral benzodiazepine receptor was localized to the mitochondria of clonal, SH-SY5Y-derived, PBR-overexpressing (and Bcl-2 overexpressing) S11 cells, as shown by western immunoblot analysis in FIG. 10 using the polyclonal antibody described above. S11 cell mitochondria were capable of taking up substantially more calcium than vector control cell mitochondria when calcium uptake studies were conducted according to described methods (Fiskum et al., 2000 Meths. Enzymol. 322:222-234; Murphy et al., 1996 Proc. Nat. Acad. Sci. USA 93:9893, 1996). The calcium uptake capacity of vector control cells was 200 μM, whereas the calcium uptake capacity of S11 cells was 400 μM.

Subcellular fractionation studies were performed as described above on the S11 cell line and analyzed for mitochondrial ETC complex IV activity (Birch-Machin et al., 1993 Meths. Toxicol. 2:58) PK-1195 binding and western blot analysis with anti-VDAC (Calbiochem, San Diego, Calif.) and anti-PBzR antibodies clearly demonstrated that overexpressed PBzR was targeted for delivery to the mitochondria in the S11 cell line. Since this is the normal subcellular localization site for the PBzR, the S11 neuroblastoma-derived, stably transfected PBzR overexpressing cell line appears to provide a useful model to define the physiological functions of PBZR.

PK-11195 treatment caused a release of cytochrome C ( determined using the method of Andreyev et al., 1998 FEBS Lett. 439:373) from S11 mitochondria upon calcium induced permeability transition (FIG. 11) to levels equivalent to cytochrome c released by vector control cells which have low levels of Bcl-2 expression. This provides apparent evidence for a functional link between PBR and Bcl-2. As shown in FIG. 12, S11 cells were protected from PK-11195 induced cell death. S11 cells and vector control cells were treated for 24 hours (4×10₄ cells per well in 96-well plates, in DMEM with 10% FCS) with a range of PK-11195 concentrations, as indicated by the values (expressed as micromolar) under each bar in FIG. 12. Cell viability was determined by propidium iodide staining cells and quantifying fluorescence with an Fmax™ plate reader (Molecular Devices, Sunnyvale, Calif.) according to the manufacturer's instructions. The percent of non-viable vector control cells increased from about 10% (0 and 20 μM PK-1102 11195) to about 40% (100 μM PK-11195). The percent of non-viable S11 cells was less than 20% at 100 μM PK-11195.

### Example 4

Caspase Activation in S11 Cells

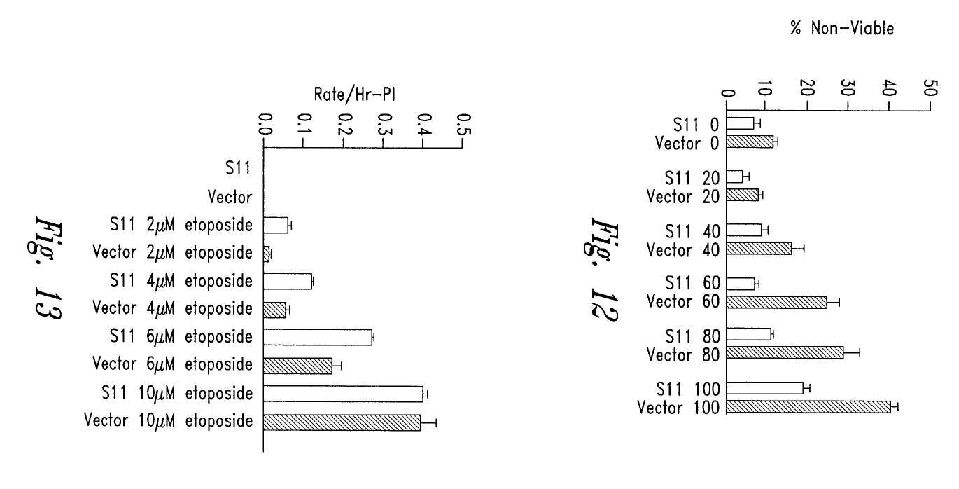

Caspases are apoptosis-activated proteases, and caspase induction in cells is often indicative that the cells are undergoing programmed cell death (apoptosis). Apoptosis can be induced by a variety of compounds, including etoposide. S11 or vector control cells were plated at 30,000 cells/well in 96 well plates and grown for 20 hours. Media was aspirated and cells treated with or without etoposide and with or without PK-11195 at the indicated concentrations in media for six hours. At this time, media was aspirated and caspase activity was measured by addition of 23 uM DEVD, a peptide that is fluorescent when cleaved by caspase 3, in PBS along with 0.03% digitonin. Fluorescence was measured in an Fmax™ 96-well plate reader (Molecular Devices, Sunnyvale, Calif.) using a kinetic assay. Total number of cells was determined using propidium iodide staining and used to normalize for total number of cells in each well. As shown in FIG. 13, at 2-6 μM etoposide, caspase activation occurred at a higher rate in S11 cells than in vector controls.

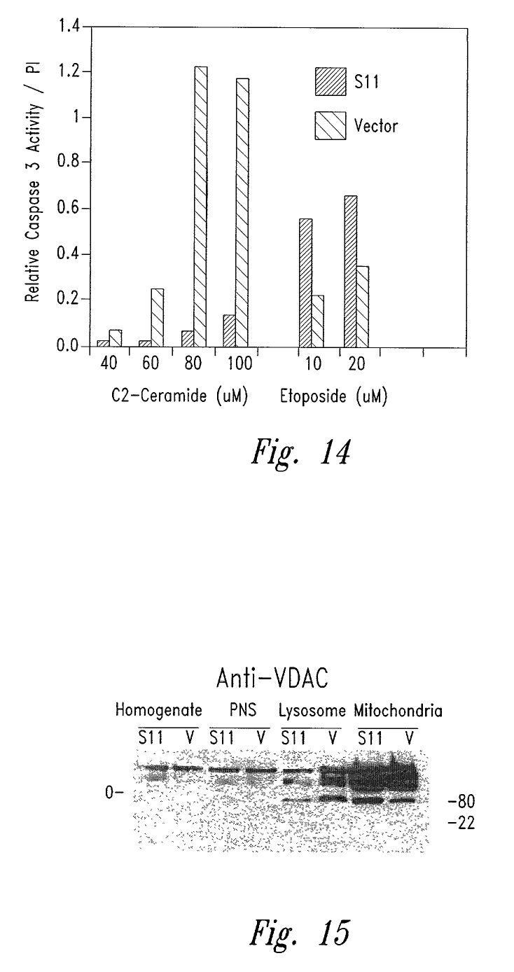

FIG. 14 illustrates the differential effects of ceramide- and etoposide-induced caspase activation in S11 cells and in empty vector-transfected controls. Etoposide and ceramide treatments were as described above. As shown in FIG. 14, and consistent with the results in FIG. 13, etoposide caused a greater activation of caspase in S11 cells than in vector controls. However, C2-ceramide caused a much greater activation of caspase in vector control cells than in the S11 cells, at concentrations of 80 and 100 μM.

### Example 5

BCL-2 Levels in Control and S11 Cells

For this series of experiments, membrane fractions were purified from S11 cells and from empty vector transfected SY5Y control cells as described above (e.g., Example 3) in order to determine the relationship between Bcl-2 and over-expression of PBZR in S11 cells. Four fractions were obtained and the degree of purification of a mitochondrial fraction was assayed using mitochondrial ETC complex IV activity and anti-VDAC (anti-voltage dependent anion channel, also known as porin) antibody detection of VDAC in western blots (FIG. 15) as described above. The results of the complex IV activity are shown in Table 2, in which complex IV activity is expressed as ΔA/min-mg:

TABLE 2
Complex IV Activity (A/min-mg) in Subcellular Fractions

| Cells | PNS | Lysosomal | Mitochondrial |
|---|---|---|---|
| S11 | 2.8 | 1.7 | 25 |
| Vector-control | 3.4 | 3.2 | 17 |

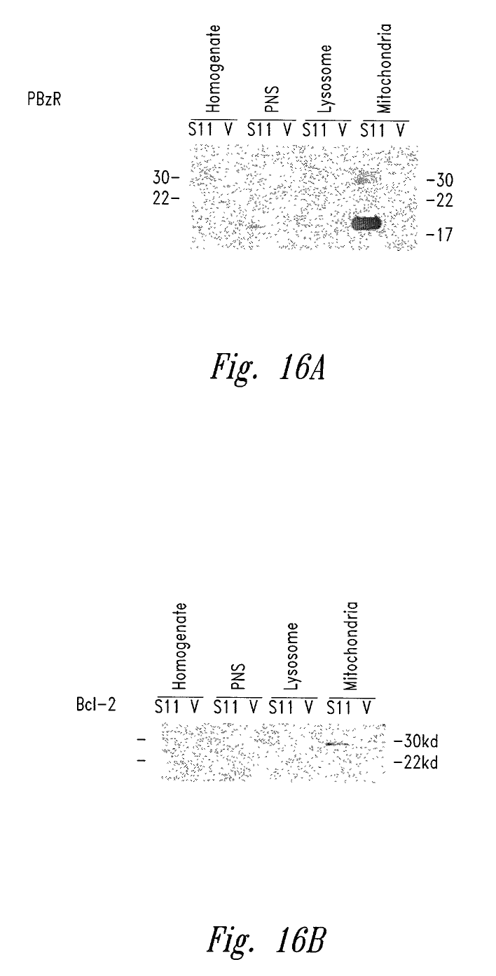

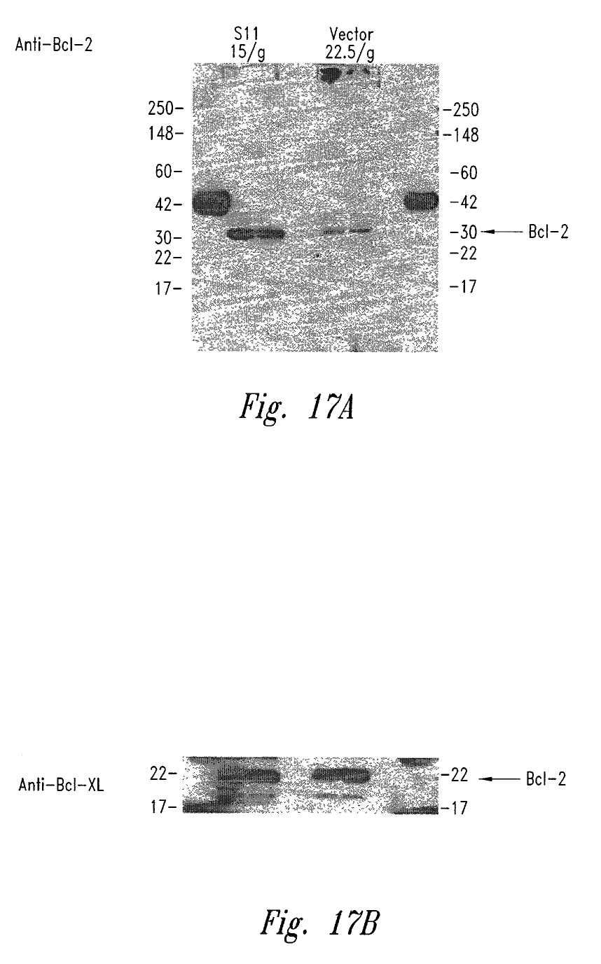

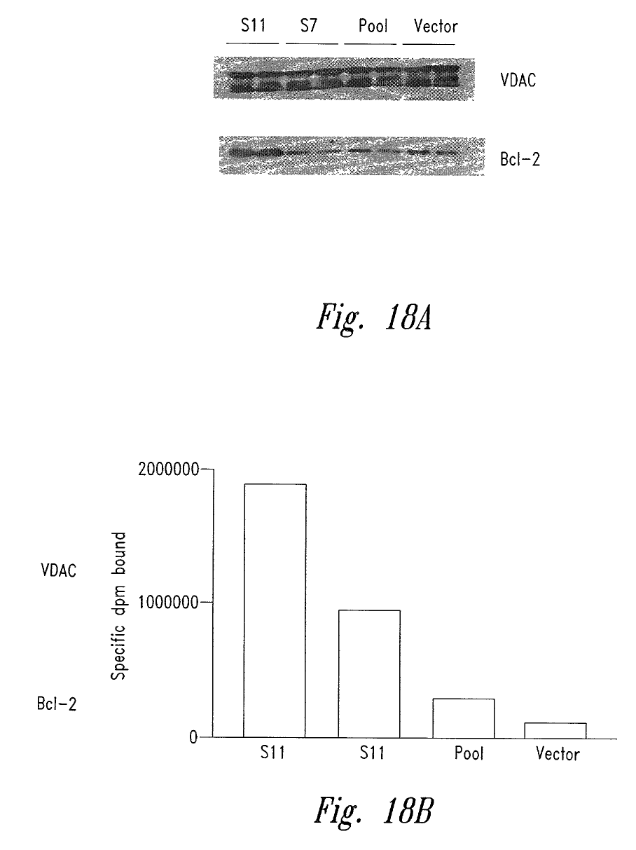

Similarly prepared subcellular fractions were used to investigate the Bcl-2 levels in S11 cells and vector control cells by western blot analysis using an anti-Bcl-2 monoclonal antibody obtained from Santa Cruz Bioscience, Inc. (Santa Cruz, Calif.) according to the supplier's recommendations. As shown in FIG. 16, overexpression of PBzR in S11 cells correlated with increased Bcl-2 levels in the mitochondrial fraction. As shown in FIGS. 17 and 18, when normalized to complex IV activity, Bcl-2 levels were increased in S11 mitochondria, but Bcl-XL levels detected by western blot analysis using anti-Bcl-XL antibody (Santa Cruz Bioscience) according to the supplier's instructions were not increased. However, the increased Bcl-2 levels in S11 cells did not appear to strictly correlate with PBzR overexpression (FIG. 18), which presents blotting data using anti-VDAC and anti-Bcl-2 to probe mitochondrial fractions normalized for mitochondrial ETC complex IV activity (FIG. 18A) that did not tightly parallel differences detected in PBzR ligand (PK-11195) binding to homogenates used for subcellular fractionation (FIG. 18B).

### Example 6

Inducible PBzR Overexpressing Cell Lines

This example describes production and characterization of inducible PBzR overexpressing cell lines, including the tetracycline-inducible PBzR overexpressing SH-SY5Y-derived cell line designated IPBR-1. Standard molecular biology reagents and methodologies were used as described, for example, in Ausubel et al. (Current Protocols in Molecular Biology, Greene Publishing, 1987); and in Sambrook et al. (Molecular Cloning: A Laboratory Manual, Cold Spring Harbor Press, 1989). SH-SY5Y neuroblastoma cells were propagated and maintained according to the supplier's recommendations (ATCC, Manassas, Va.) and the tetracycline repressor was stably integrated into the cells using the pcDNA6/TR vector (Invitrogen, Carlsbad, Calif.) and blasticidin selection according to the instructions accompanying the vector. Tet^{R} clones were then stably transfected with a modified pcDNA4/TO vector (Invitrogen) into the multiple cloning site of which the full length coding sequence for peripheral benzodiazepine receptor (GENBANK Accession No. NM_{—}000714; 1991 Eur. J Biochem. 195:305-311), amplified by PCR from a human placenta cDNA library, had first been ligated using the supplier's protocols. The resulting construct contained the PBzR coding sequence under the control of a tetracycline-regulated promoter. Colonies were selected for zeocin resistance and individual clonal populations were isolated.

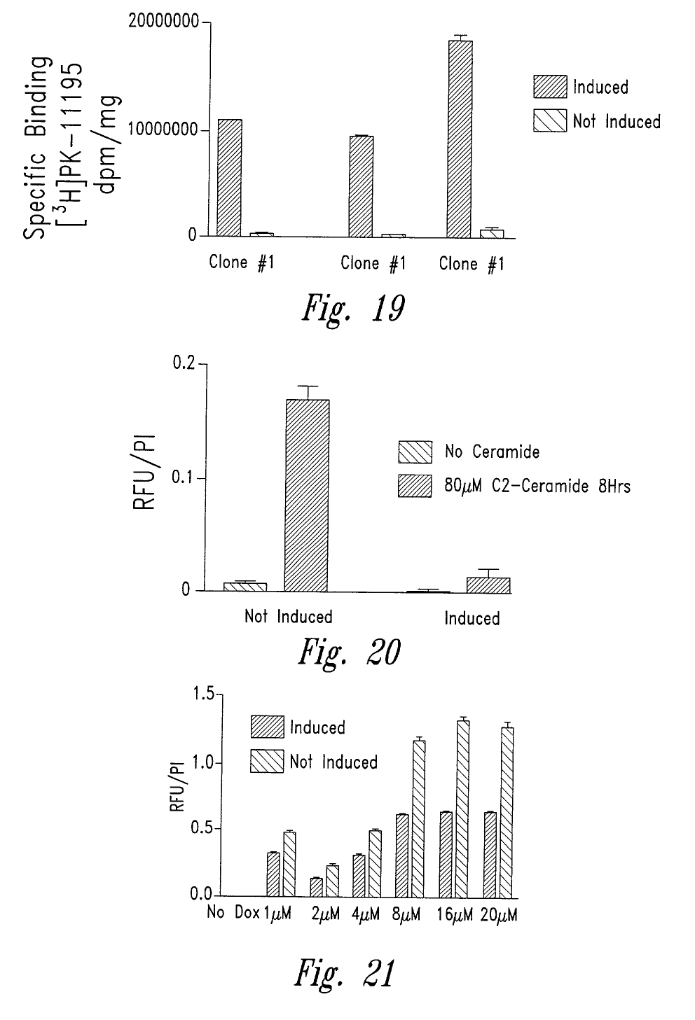

Double-labeling immunofluorescence analysis of tetracycline-induced transfectants with anti-PBzR antibodies (see Example 3) and anti-cytochrome c antibodies (Santa Cruz Biosciences, Inc., Santa Cruz, Calif.) showed PBzR that had localized to mitochondria; mitochondrially localized PBZR was not frankly apparent in tetracycline-uninduced cells. Three isolated clones were analyzed for tetracycline-inducible PBzR expression by comparing tritiated PK1115 binding to induced and uninduced cells using the procedure described above for FIG. 5 (FIG. 19) FIG. 19 shows markedly increased PBzR ligand binding to each of three clonal populations of induced cells relative to the corresponding uninduced cells.

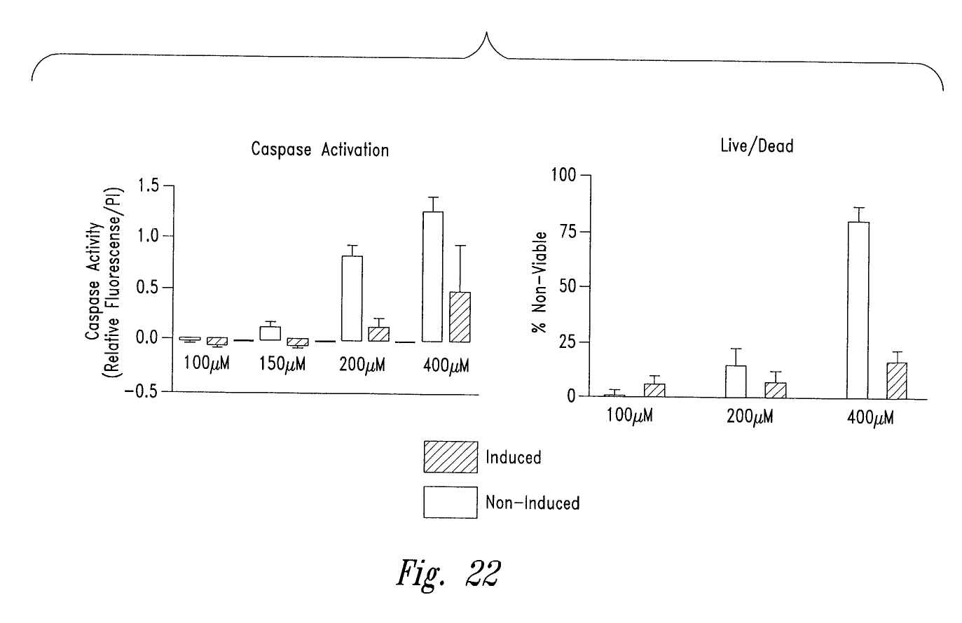

The effect of tetracycline induced PBzR overexpression on apoptogen-driven (C₂-ceramide, 80 μM, 8 hrs) caspase activation was examined in a selected, oligoclonal tetracycline-inducible PBzR overexpressing SH-SY5Y-derived cell line (FIG. 20). Inducible PBzR overexpression in these cells promoted a state of resistance to ceramide-stimulated caspase activation (see Example 4 for treatments), relative to uninduced cells (FIG. 20). Induction of PBzR overexpression similarly moderated the degree of caspase activation in one of the inducible clones, a tetracycline-inducible PBZR overexpressing SH-SY5Y-derived cell line designated IPBR-1, following exposure to another apoptogen, doxorubicin (4 μM, 8 hrs) at the indicated concentrations (FIG. 21). As shown in FIG. 22, induction of PBzR overexpression in IPBR-1 cells correlated with resistance to caspase activation in response to a third apoptogen, the NO and O₂ donor SIN-1 (3-morpholinosydnonimine, HCl; Calbiochem; 100-400 μM). The caspase response also correlated with cell viability determinations (FIG. 22), consistent with presumed caspase roles in apoptotic cascades. Conversely, no reduction in caspase activation was detected in tetracycline-induced IPBR-1 PBzR overexpressers following exposure to another apoptogen, thapsigargin, suggesting a distinct mechanism for initiation of apoptosis by this agent. Western blot analysis of IPBR-1 mitochondrial fractions isolated on metrizamide gradients and normalized on the basis of mitochondrial ETC Complex IV activity (as described above) confirmed a dramatic increase in PBzR expression. No discernible change in Bcl-2 expression levels, however, accompanied the induction of PBzR overexpression.

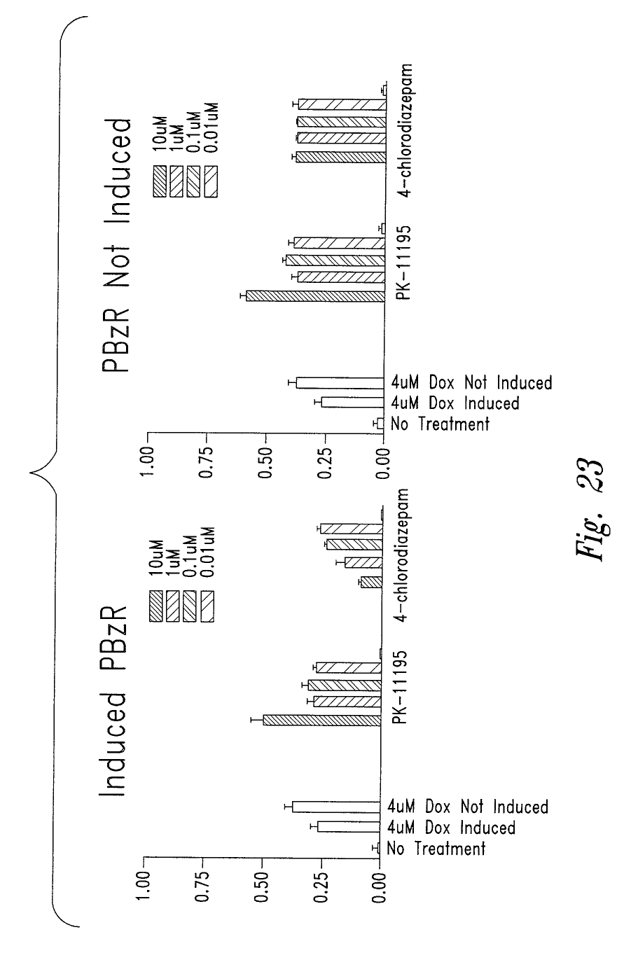

Activation of caspase in response to treatment with the chemotherapeutic agent doxorubicin (Sigma, St. Louis, Mo.) was also compared in tetracycline induced and uninduced IPBR-1 cells that had been treated with one of the PBzR ligands PK-11195 or 4-chlorodiazepam (FIG. 23), or with other PBzR ligands. Unlike the other PBzR ligands, 4-chlorodiazepam appeared to confer on induced but not on uninduced cells, and in a dose-dependent fashion, a protective effect against the doxorubicin induced activation of caspase (FIG. 23). Accordingly, the present invention provides the unexpected finding that peripheral benzodiazepine ligands may be distinguished on the basis of whether they preferentially alter apoptosis, and whether they preferentially alter a mitochondrial function. Data also indicate that benzodiazepine-related PBR ligands can act as effective anti-apoptotic agents, which may be of benefit in the treatment of certain neuropathologies and/or in chronic inflammatory conditions.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of screening for an agent that binds a peripheral benzodiazepine receptor, comprising the steps of:
(a) contacting a sample comprising a mitochondrion from a cell that overexpresses a peripheral benzodiazepine receptor with a peripheral benzodiazepine receptor ligand and a candidate agent; and
(b) detecting a level of binding of the peripheral benzodiazepine receptor ligand to the peripheral benzodiazepine receptor, relative to a level of binding in the absence of candidate agent, and therefrom identifying an agent that binds a peripheral benzodiazepine receptor.

2. A method according to claim 1, wherein the sample comprises an intact cell that overexpresses a peripheral benzodiazepine receptor.

3. A method according to claim 2, wherein the cell is a permeabilized cell.

4. A method according to claim 2, wherein the cell is a neuronal cell.

5. A method according to claim 1, wherein the peripheral benzodiazepine receptor is a mitochondrial peripheral benzodiazepine receptor.

6. A method according to claim 1, wherein the peripheral benzodiazepine receptor ligand is detectably labeled.

7. A method according to claim 1, wherein the candidate agent is an agonist of the peripheral benzodiazepine receptor ligand.

8. A method according to claim 1, wherein the candidate agent is an antagonist of the peripheral benzodiazepine receptor ligand.

9. A method according to claim 1, wherein the peripheral benzodiazepine receptor ligand is selected from the group consisting of PK-11195, 4-chlorodiazepam, DAA1106 and DAA1097.

10. A method of screening for an agent that alters mitochondrial function, comprising the steps of:
(a) contacting, in the presence of a candidate agent:
(i) a sample comprising a mitochondrion from a cell that overexpresses a peripheral benzodiazepine receptor, and
(ii) a peripheral benzodiazepine receptor ligand;
(b) evaluating at least one mitochondrial function in the sample; and
(c) comparing the mitochondrial function to a mitochondrial function detected in the absence of the candidate agent, and therefrom identifying an agent that alters mitochondrial function.

11. A method of screening for an agent that alters mitochondrial function, comprising the steps of:
(a) contacting, in the presence of a candidate agent:
(i) a sample comprising a mitochondrion from a cell that overexpresses a peripheral benzodiazepine receptor,
(ii) a compound that alters mitochondrial membrane potential, and
(iii) a peripheral benzodiazepine receptor ligand;
(b) evaluating at least one mitochondrial function in the sample; and
(c) comparing the mitochondrial function to a mitochondrial function detected in the absence of the candidate agent, and therefrom identifying an agent that alters mitochondrial function.

12. A method according to either claim 10 or claim 11 wherein the mitochondrial function is evaluated by determining mitochondrial membrane potential.

13. A method according to either claim 10 or claim 11 wherein the mitochondrial function is evaluated by detecting a level of apoptosis.

14. A method according to either claim 10 or claim 11 wherein the mitochondrion is present within an intact cell.

15. A method according to either claim 10 or claim 11 wherein the mitochondrion is present within a permeabilized cell.

16. A method according to either claim 10 or claim 11 wherein the mitochondrion is present within a cell that overexpresses a peripheral benzodiazepine receptor.

17. A method according to either claim 10 or claim 11 wherein the candidate agent is an agonist of the peripheral benzodiazepine receptor ligand.

18. A method according to either claim 10 or claim 11 wherein the candidate agent is an antagonist of the peripheral benzodiazepine receptor ligand.

19. A method according to either claim 10 or claim 11 wherein the peripheral benzodiazepine receptor ligand is selected from the group consisting of PK-11195, 4-chlorodiazepam, DAA1106 and DAA1097.

20. A method according to either claim 10 or claim 11 wherein the cell is a neuronal cell.

21. A method of screening for an agent that alters a mitochondrial function, comprising the steps of:
(a) contacting, in the presence of a candidate agent:
(i) a cell that overexpresses a peripheral benzodiazepine receptor,
(ii) a chemotherapeutic agent, and
(iii) a peripheral benzodiazepine receptor ligand;
(b) detecting a level of Bcl-2 binding to a Bcl-2 ligand in the cell; and
(c) comparing the level of binding to a level of Bcl-2 binding to a Bcl-2 ligand detected in the absence of the candidate agent, and therefrom identifying an agent that alters a mitochondrial function.

22. A method according to claim 21 wherein the cell overexpresses Bcl-2.

23. A method according to claim 21 wherein the cell is a neuronal cell.

24. A method according to claim 21 wherein the cell is permeabilized.

25. A method according to claim 21 wherein the candidate agent is an agonist of the peripheral benzodiazepine receptor ligand.

26. A method according to claim 21 wherein the candidate agent is an antagonist of the peripheral benzodiazepine receptor ligand.

27. A method according to claim 21 wherein the peripheral benzodiazepine receptor ligand is selected from the group consisting of PK-11195, 4-chlorodiazepam, DAA1106 and DAA1097.

28. The method of claim 21 wherein the mitochondrial function is evaluated by determining mitochondrial membrane potential.

29. The method of claim 21 wherein the mitochondrial function is evaluated by detecting a level of apoptosis.

30. A method according to claim 29 wherein the step of comparing the level of apoptosis is by an assay determination selected from the group consisting of vital dye staining of the cell, cell blebbing, caspase activity, DNA fragmentation, cytochrome c release and annexin binding to the cell.

31. The method of any one of claims 1, 10, 11 or 21 wherein the cell that overexpresses a peripheral benzodiazepine receptor is capable of being induced to express the peripheral benzodiazepine receptor.

32. A method for identifying a peripheral benzodiazepine receptor ligand that preferentially alters apoptosis, comprising:
(a) contacting,
(i) a peripheral benzodiazepine receptor ligand,
(ii) a cell that is capable of being induced to overexpress a peripheral benzodiazepine receptor, and
(iii) an apoptogen, under conditions and for a time sufficient to induce apoptosis in said cell; and
(b) comparing (i) a level of apoptosis in said cell that has been induced to overexpress a peripheral benzodiazepine receptor, to (ii) a level of apoptosis in said cell that has not been induced to overexpress a peripheral benzodiazepine receptor, wherein a decreased level of apoptosis in said cell that has been induced relative to the level of apoptosis in said cell that has not been induced indicates that the peripheral benzodiazepine receptor ligand preferentially alters apoptosis.

33. A method for identifying a peripheral benzodiazepine receptor ligand that preferentially alters a mitochondrial function, comprising:
(a) contacting,
(i) a peripheral benzodiazepine receptor ligand,
(ii) a cell that is capable of being induced to overexpress a peripheral benzodiazepine receptor, and
(iii) an agent that alters a mitochondrial function, under conditions and for a time sufficient to induce at least one altered mitochondrial function in said cell; and
(b) comparing (i) a level of at least one mitochondrial function in said cell that has been induced to overexpress a peripheral benzodiazepine receptor, to (ii) a level of said at least one mitochondrial function in said cell that has not been induced to overexpress a peripheral benzodiazepine receptor, wherein a decreased level of the mitochondrial function in said cell that has been induced relative to the level of the mitochondrial function in said cell that has not been induced indicates that the peripheral benzodiazepine receptor ligand preferentially alters a mitochondrial function.

34. The method of either claim 32 or claim 33 wherein the cell that is capable of being induced to overexpress a peripheral benzodiazepine receptor is of neuronal origin and the peripheral benzodiazepine receptor ligand is neuroprotective.

35. A cell line modified to express at least about ten-fold more peripheral benzodiazepine receptor protein than a parental cell line from which it is derived, and which overexpresses Bcl-2.

36. The cell line of claim 35 which is modified to express at least about three-fold more Bcl-2 protein than a parental cell line from which it is derived.

37. The cell line of claim 35 wherein the parental cell line is a neuroblastoma cell line.

38. The cell line of claim 35 that is designated S11.

39. A cell line modified to be capable of being induced to express at least about ten-fold more peripheral benzodiazepine receptor protein than a parental cell line from which it is derived.

40. The cell line of claim 39 which is modified to express at least about three-fold more Bcl-2 protein than a parental cell line from which it is derived.

41. The cell line of claim 39 wherein the parental cell line is a neuroblastoma cell line.

42. The cell line of claim 39 that is designated inducible PBzR overexpressing SH-SY5Y-derived cell line or IPBR-1.
